(19) United States
(12) Patent Application Publication
(10) Pub. No.: US 2007/0185751 A1
(43) Pub. Date: Aug. 9, 2007

(54) Drug life cycle management system

(76) Inventor: Ramon J. Dempers, Alpharetta, GA (US)

Correspondence Address: BIDDLE & ASSOCIATES, 6300 POWERS FERRY ROAD, SUITE 600-183, ATLANTA, GA, 30339, US

(21) Appl. No.: 11/652,849

(22) Filed: Jan. 11, 2007

Related U.S. Application Data
(60) Provisional application No. 60/758,170, filed on Jan. 11, 2006.

(51) Int. Cl. G06F 9/44; G06Q 10/00; G06F 17/00; G06F 7/00

(52) U.S. Cl. 705/7; 705/2; 235/376; 707/104.1

(57) ABSTRACT

A system and method are provided for collecting all data relevant to a drug product throughout the life cycle of the drug product(life cycle data), applying a common rules set to determine and initiate responsive action and storing the collected data into a centralized data repository. The system and method allows for the monitoring of collected data to identify trigger events and to initiate predetermine actions based upon predetermined rules. The system is configured to collect data and assemble it in a centralized data repository. Further, the system is configured to analyze data in accordance with predefined rules and determine current status of one or more factors.

### CROSS-REFERENCE TO RELATED APPLICATION(S)

This application claims priority with respect to U.S. Provisional Application having Ser. No. 60/758,170 filed on Jan. 11, 2006, the disclosure of which is hereby incorporated herein by reference.

### TECHNICAL FIELD OF THE INVENTION

The present invention is generally related to management and generation of information connected with and during the life cycle of a product produced by a life science organization (LSO), such as a drug, medical device, biologic and/or human tissue product (collectively referred to as LSO product). More particularly, the present invention is directed to a system and method for collecting relevant to a LSO product from all entities involved in the life cycle of the LSO product.

### BACKGROUND

Bringing a drug, medical device, biologic and/or human tissue product (collectively referred to as LSO product) to the marketplace is a complex, time consuming and very expensive process. Aside from the burdens that the process of research, development, clinical trials, medical safety; quality control and assurance, production, distribution and marketing and post approval compliance monitoring impose on a life science organization (LSO) that sponsors a LSO product and attempts to bring it to market, or a LSO marketing manufacturing and managing an approved LSO, the regulatory reporting and audit requirements imposed on the life science organization adds a further, and staggering, burden on a life science organization involved in bring a LSO product to the market place, monitoring it throughout the production and post-production part of the life cycle, this includes ongoing stability monitoring, LSO safety adverse events monitoring and reporting, until the LSO product expires and is recalled or recalled for reasons of safety identified in on-going stability monitoring.

With reference to FIG. 1, the life cycle of a LSO product extends through several phases and business disciplines, from research 10 to development 12, through clinical trials 14, on to production and distribution 16 and then to the marketplace 18 on to post manufacture monitoring compliance and management. Each phase of this life cycle involves multiple organizations and/or functional entities and multiple personnel, all focused on specific aspects and/or issues related to the Drug product.

FIG. 2A is a diagram depicting the various business entities (organizations, departments or functional entities) that are involved in bringing a drug (or other LSO product) to the marketplace and subsequently, monitoring and managing, post approval drugs and devices. These business entities 201 may be a research department 220; a development department 222; a clinical management department 224; production department 226; quality assurance/control department 236; medical affairs department 234; legal affairs department 232; regulatory affairs department 230; human resources department 238; marketing department 239 and laboratory management department 240. Each of these business entities/departments may be a part of the LSO 28 or may be an independent organization contracted by the LSO 28 to carry out particular work.

FIG. 2B shows a diagram depicting some of the various organizations or functional entities that are typically involved at each stage of the drug life cycle. With reference to FIG. 2B, during the research phase 10, the research organization(s) 220 are involved in conducting research and collecting research data. Similarly, throughout the life cycle of the LSO product, multiple administrative functions are carried out by the various administrative business entities (generally shown as administrative 239). Regulatory affairs 230 is involved in assuring that all regulatory issues/requirements are met and that compliance is otherwise in order. Legal affairs 232 is involved in assuring that legal requirements are met. Medical affairs 234 is involved in monitoring the drug safety information for the drug, tracking and reporting all adverse events (expected and non-serious; unexpected and non-serious; unexpected and serious; expected and serious or fatal, while quality department 236 is involved in quality control and assurance to ensure quality standards are established and adhered to during the research phase. Human resources 238 is involved with personnel and their qualifications and training records relating to personnel involved in the research phase.

Typically regulatory affairs 230, legal affairs 232, medical affairs 234, quality department 236 and human resources 238 functions are carried out directly, as an in-house operation, by the LSO 28. However, any one or more of these business entities/functions may be carried out by an external, independent organization contracted by the drug sponsor 28. For example, at the development phase 12 a development department 222 works to develop a proposed Drug product. Data is generated and collected by this the development department 222. Similarly, all the administrative entities/functional operations of administration 239 (example: 230, legal affairs 232, medical affairs 234, quality department 236 and human resources 238 are carried out during the development phase 12). Each of these entities/operations will generate and collect data relevant to the development phase. At the clinical trials phase 14, clinical testing management 24 generates and collects data relevant to clinical trials. The other entities/operations 230-238 are also carried out during this phase and relevant data generated/collected. At the production/distribution phase 16 the production/manufacturing organization 226 generates and collects data related to the manufacture and distribution of the approved Drug product. The other entities/operations 230-238 are also carried out during this phase and relevant data generated/collected. Data may include documents and files (hard copy and/or electronic formats), as well as other information relevant to a Drug product.

As noted above, throughout the life cycle of a Drug product, each organization/entity involved generates and collects data/information relevant to the new and existing Drug product/aspects thereof. This information is important and necessary for proving the LSO products efficacy, regulatory compliance, as well as legal compliance and other requirements and objectives. It is also very important as a potential source for determining the reason(s) for any adverse events reported once the LSO product has reached the marketplace.

Each business entity 200 involved in the process generates and/or collects and stores data/information related to their specific work/activities related to the Drug product. This data/information is stored in accordance with the Standard Operating Procedures (SOP) of the particular organization and/or individual involved. Not all business entities 200 abide by the same or similar storage/archiving scheme (location, format, access, processing rules, etc.).

The result is that relevant data pertaining to a particular LSO product-generated/collected during the drug life cycle—from the Research & Development phase to marketplace phase—is collected and stored in multiple disparate places (or separate SILOs of information). Further this data is often embodied in multiple formats, from printed hard copies to various electronic file formats stored on various independent storage media, such as, for example, compact disk (CD-ROM), digital versatile disks (DVD), magnetic tape; some searchable, some not. This disparity in how and where each organization manages (generate, collects, analyzes and/or stores) data relevant to a LSO product makes subsequent access of such relevant data a near nightmare, particularly as time goes on.

Historically, data management systems have been purchased and implemented by departments/business entities according to the organizational chart and the specific needs of that/each department. Each has the authority to buy and implement (or to not buy and implement) technology or other solutions based on their department's needs only. As a result the company develops “silos” or stove pipes of data each managed in isolation from other data in the company, many data management functions/systems overlap, go unused or are non-existent. From a higher-level process perspective, many gaps exist. The layers of complexity grow thicker as the life science organization expands, interfaces with outside contractors or organizations, acquires other companies, changes their organizational plans or in other ways increase the number of nonintegrated systems.

The result is that the typical life science organization is finding that they do not have a clear understanding across the entire organization of the status of a particular product from all perspectives; quality; safety; compliance; documentation and other perspectives, thus increasing the risks to the company from a regulatory and patient safety perspective. While the management and validation of these highly complex environments is expensive, prone to system failure and prone to user error and even deviations in quality of the product, number into the many plant and value-chain applications. For all but the largest budgets/companies, such expenses are just not an option. The result is that data relevant to a given LSO product will be scattered among multiple disparate data management systems and storage locations, often inaccessible to all but the persons of organization/entity directly responsible for generating and maintaining it.

Any attempt to apply common information technology department standards to the variety of disparate departments/systems is difficult and costly. Finally, the portion of the budget dedicated just to maintaining older legacy, narrow applications is growing, making it more difficult to adopt new systems that are be broader and more accessible.

FIG. 3A and FIG. 3B are diagrams describing the typical manner in which data is generated, collected and stored by each business entity 200 (222, 224, 226, 228, 230, 232, 234, 236, and 238) that are involved during the life cycle of a Drug product. With reference to FIG. 3A it can be seen that it is quite common for each entity to have or otherwise employ data management systems and practices that are separate and distinct from the data management system used by each of the other business entities 200 involved in the LSO product life cycle. Once the data is collected and stored away, the data is stored into these SILOS and is long forgotten about after several years despite is the data's on-going usefulness to on-going drug monitoring, trending and analysis and possibly even the discovery of new therapies and treatments for the drug. Finding and/or retrieving this information from among the data SILOS of these various business entities 200 is difficult and time consuming, as it often involves manual search and identification efforts to locate and search.

The data management tools used by the various organizations involved in the management of an LSO product are typically different, although they allow for the same or similar operations to be carried out. Where these systems incorporate or are otherwise software based, regulations may require that these systems be validated and re-validated each and every time changes or updates are made to the software.

Because of the disparity in data management tools and efforts at each stage of the drug life cycle, life science organizations are faced with several problems, including, but not limited to: 1) Meeting regulatory agency audit requirements in the LSO, when the organization is working on manual and or semi-automated systems, access and collection of relevant information is cumbersome (onerous). The life science organization must expend massive man power and expense to assemble relevant data for generating and filing reports necessary for regulatory compliance. For smaller organizations, this often means that personnel, who would otherwise be working on core functions, are distracted for significant periods of time with the mind numbing data collection and assembly tasks. For other smaller organizations, the burden can be too much to fulfill and they are then at risk of severe penalties, including heavy fines, product withdrawal and even business closure. The inability to quickly and completely gather the necessary relevant information to meet regulatory requirements imposes a great burden on life science organizations. 2) Analyzing Adverse Events—When adverse events are reported after a drug has been approved, the key to determining why an adverse event has occurred and what the solution may be often lies in a combination of data collected during the research/development, clinical trials and the post-approval quality/production/distribution phase of the drug life cycle and the information reported in the new adverse event. The inability to quickly and completely gather necessary relevant information for purposes of a thorough investigation of reported adverse events imposes a great burden on the life science organization and makes prompt corrective action difficult. It also makes the possibility of proactive steps to avoid an adverse event nearly, if not completely, impossible; and 3) Validation—Where data management tools used in the life science industry incorporate or are otherwise based on software, strict rules are applied with respect to validation (intended use and function) of the software. Where there multiple separate data management tools that incorporate software, the validation process must be conducted each time there is a change or update to the software. This is a time consuming and expensive process, and gets more expensive and time consuming when multiple systems are at issue. If the company fails to maintain a “Validated State” in compliance with Regulating Authority Laws, it faces fines and other penalties. The maintenance of the Installation Qualification (IQ); Operational Qualification (OQ); Performance Qualification (PQ); Master Validation Plan; Master Validation Index and traceability matrix often costs three times that of the software it is validating.

Generating documents/reports/promotional materials—Many documents are typically generated by the LSO through out the life cycle of a drug product. Great effort is required to assure that these documents are accurate, complete and do not run afoul of legal or regulatory guidelines. This generally requires the involvement of personnel from the multiple business entities/departments etc., that each utilize their own separate and distinct data management system for generating and handling data/information related to a given drug product. Naturally, the process of insuring that all relevant parties/entities review and provided necessary input on documents before they are released or otherwise distributed for quality control/quality assurance/medical/promotional/regulatory purposes is a cumbersome, time consuming effort that has no provisions for assuring that all departments have reviewed the document and made sure it properly addresses its topic before it is released to the consumer of information. Trigger Events. Required Actions and Deadlines—Clearly just gathering the necessary information to complete regulatory reporting obligations is a significant burden in and of itself. However, meeting the reporting requirements in a timely manner is made even more difficult since the occurrence of certain unplanned events, such as adverse events reported from the marketplace, can trigger a requirement to file a report or take certain other action within a set timeframe. This timeframe can be on the order of only a few days or even hours. For the typical life science organization, significant effort is put into knowing and understanding when regulations require reporting or other action. That said, the regulations are voluminous and keeping track of all rules, trigger events and required actions is a daunting task. For a smaller organization, it is a near administrative nightmare and can and often results in severe penalties or even closure or bankruptcy of the business, due to the financial burden placed on them to demonstrate compliance and the safety of their LSO product.

Testing and Analysis: Only as good as the people and equipment—During the lifecycle of a drug product, particularly after it has been approved, periodic testing and analysis of samples of drug components, such an active pharmaceutical ingredients (API) that comprise the drug product, must be made to ensure that the manufactured LSO product meets or continues to meet specific predetermined criteria. This periodic testing and analysis reduces the chance that defective or nonconforming LSO product is ever placed into the marketplace. However, test results are only as good and the test equipment used to conduct the tests and the people who operate the equipment.

The Testing and analysis of compounds/elements (LSO product components) that comprise a LSO product occurs at various points during the lifecycle of the drug product. For example, during the manufacture of a LSO product samples are taken before each production run from each batch of one or more of the elements/compounds that will comprise the drug product. The samples are then tested and analyzed to make certain that they meet specifications. Once the test and analyses are complete, a certificate of analysis (CofA) is issued if the sample does in fact meet specified criteria. If the test and analysis of a sample of a drug component fails to meet specified criteria, no CofA is issued and the sampled batch is then rejected or otherwise discarded by the manufacturer.

Another point at which testing and analysis of a sample of a drug component may be conducted is after the manufactured LSO producthas been distributed and placed into the marketplace. Such a test may occur months or even years after the LSO product has reached the marketplace. The purpose of such a test and analysis is to determine whether or not a drug component remains stable and otherwise continues to meet specifications over time. As drug components may or may not remain stable over time, the effectiveness and or safety of a LSO product may change after it has been distributed into the marketplace and been sitting on distributors; retailers and in consumers medicine cabinets for a period of time. When a drug component becomes unstable or otherwise fails to meet specified criteria, LSO product already in the marketplace may be/become unsafe for use.

The accuracy of testing and analysis is highly contingent upon the proper functioning of equipment used to conduct the specific test/analysis, as well as the personnel who operate the equipment to conduct tests. More particularly, equipment that is not properly calibrated can result in inaccurate test results. Further, personnel who have not been trained to operate a particular piece of test equipment, or who are not current with relevant continuing professional education (CPE) requirements, introduce the significant possibility that the tests they conduct are not run properly, and thus that the test results are inaccurate and can not be relied upon.

Where it is determined that a drug component fails to continue to meet specified criteria, a complicated quality management process is initiated called an Out of Specification (OoS) deviation or non-conformance. This triggers the start of an investigation into the failure, and once the root cause for the failure is established a corrective action and preventative action (CAPA) is executed to determine if the OoS can be remedied or if the production run must be stopped and product destroyed as may be required by regulatory authorities best practices. The corrective action in tern might require the initiation of one or more changes to procedures, equipment or require additional personnel training. Change management follows a process, in which the risk of the required change is evaluated from several perspectives including but not limited to a patient safety; regulatory; cost; production and other perspectives. This evaluation is conducted based on the data gleaned during the investigation of the deviation and data collected during other similar deviations. If there is agreement that the change is required, the change effort is assigned to various individuals through what are called assessments. The work is performed and the change control is completed and signed-off. The preventative action part of CAPA then requires that the company periodically remember to check the efficacy of the change to ensure the change has been effective for final closure of the initial OoS; Investigation; corrective action; change control and preventative action.

Results of periodic testing of drugs may be tainted when testing equipment is not calibrated or equipment operators not trying to or otherwise current on their continuing professional education (CPE). The result is that a CofA may be issued even though the test compound is not actually to specification. Once the CofA is issued, the manufacturing process continues and, in the case of a flawed test, the production of the LSO product continues even though an element/compound used to make the LSO product does not actually conform to specifications. This raises the risk that the drug product, once ingested by a consumer, will be ineffective, or worse, cause the consumer to have adverse reactions resulting in illness and in some cases death.

Clinical trials: Swaying the Outcome—Once research and development efforts yield a possible a new LSO product candidate after exhaustive testing and research using non-human subjects such as animals and data model and statistics, the new LSO product is submitted to clinical trials to determine whether or not it actually performs safely and effectively within a population of clinical trial subjects (trial subjects). Before clinical trials can ever begin, regulatory permission must be requested and obtained. If permission is obtained the trial sponsor (the party requesting the clinical trial, generally the drug sponsor 28) conducts three sets of Clinical Trials called Phase 1; Phase 2 and Phase 3 (in some cases a Phase 0 may also be conducted). These trials, through the collection and analysis of trials data allows for the development of such things as product documentation as well as proof of the effectiveness of the drug.

If permission is granted by the regulatory authority, clinical trials are conducted in accordance with a predefined hypothesis and protocol. These trials can be carried out by the sponsor or by a contract research organization (CRO) that is independent of the trial sponsor. One of the first steps in the clinical trials is the enrollment of trial subjects or trial participants. As trials progress the CRO monitors the trial subjects and collects relevant data (trials data). Once clinical trials are completed, the collected data is analyzed and reported to the trials sponsor.

For the duration of clinical trials of a proposed drug, data (trial data) is collected based upon the trials protocol. The protocol essentially sets out all the factors and/or variables that should be monitored, noted, collected and/or otherwise obtained when examining trial subjects.

As a clinical trials progresses, collected data may show trends that can/may allow for prediction/forecasting of the ultimate outcome of the clinical trials. Access to clinical trials data during the duration of clinical trials could present people/entities with a vested interest in the outcome of the clinical trials with the opportunity to attempt to sway or otherwise influence the outcome of the clinical trials by suggesting/making changes in the trials plan so that a suitable/desired outcome will result. While a desired outcome may be achieved, such changes in the trials plan skew the test results in such a way that true understanding of cause and effect can not be made. Further, such changes in trials plan are prohibited under many regulatory schemes.

After the tests are conducted the company must submit a New Drug Application (NDA) in a specific format (called electronic Common Technical Document (eCTD)) if it is electronic or and Common Technical Document (CTD) if submitted in paper form. The NDA is a voluminous application to request permission to market the Drug. The application must include, among other things, all supporting data and information pertaining to the research and development efforts that led to identifying the new drug product, including but not limited to all Quality; Clinical; Medical; Safety and statistical data generated/gathered during the research, development and clinical phases of the life cycle. This support information is typically dispersed among multiple business entities 200 and/or storage places and originates from multiple persons within these organizations. For the majority of life science companies (LSOs), assembling this information alone is a monumental, time consuming effort, since the vast majority of life science organizations manually manage the collection and storage of data and other information related to research and development efforts. Once completed, the application is submitted to relevant regulatory authorities, such as, for example, the United States Food and Drug Administration (FDA).

Inability to Access Data Inhibits Pro-Active Problem Avoidance Measures—As an example of how the prior art functions, consider the scenario where a drug has successfully gone through clinical trials and is subsequently approved for introduction into the market place by relevant regulatory agencies. During the clinical trials data was collected on each trial participant. Information such as, for example, age, weight, race, health history and current vital health statistics were collected via regular examinations of trial participants during the trial period.

After the drug is introduced into the marketplace, reports are received indicating that users of the drugs are experiencing loss of skin from their bodies in large sheets. The reports are received by company management. However, as most/all data relevant to the drug at various stages of the drug life cycle is stored in various, unrelated places and in various disparate and non-searchable formats (papers stored in file boxes in storage facility) there is no way to quickly determine the problem or how it may be connected to the drug. With this scenario in mind, further consider that a review would show that all the people reporting loss of skin after using the drug are of Asian descent.

Further consider that a review of clinical trial records/data would show that absolutely zero trial participants were of Asian decent. If there is a possible link, the prior art does not know and can not give company management any rapid and accurate guidance. Further, it can not provide management with quick/easy access to all information relevant to the drug that has been collected thus far. To gain any further insight into the problem will require countless hours of searching and manually retrieving data/file boxes from any number of various locations and computer systems and dispersed databases. It will then require substantial manual review of documents before anyone will ever be able to make a connection between circumstances related to clinical trials and the currently reported problem.

If there is a link, what actions might need to be taken? The prior art can not tell you. Should the product packaging information be revised to provide a warning to persons of Asian descent about possible adverse effects that could result from use of the drug? The prior art does not know and can not recommend such action without an immense effort on the part of all persons assigned to this investigation. While this effort is underway, the regulating agency often has it within it's power to require the company to cease and desist from any and all further manufacture, distribution and sale of their drug product. In short, the regulatory authority can effectively shut the company down.

Thus, a heretofore unaddressed need exists in the industry to address the aforementioned deficiencies and inadequacies. The features and advantages of the present invention will be apparent from the following description of the invention. The accompanying drawings, listed herein-below, are useful in explaining the invention.

### SUMMARY OF THE INVENTION

The present invention provides a system and method of managing data relevant to a medical product during the life cycle of the medical product. More particularly, the present invention is related to a system and method of collecting data related to a drug product, analyzing the data in accordance with a common set of rules and adding the data to a central data repository.

In one implementation of the invention a system is provided that includes a controller configured to analyze LSO product data in accordance with a common set of rules and to store the LSO product data into a central data repository.

In a further implementation of the invention a method of collecting data is provided that includes the steps of receiving data related to a drug product, analyzing the data in accordance with a common set of rules and storing the data to a central data repository.

Other systems, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a diagram generally depicting the life cycle of a drug product.

FIG. 2A is a diagram generally descriptive of the entities and/or functional departments or operations of a Drug Sponsor 28.

FIG. 2B is a diagram generally depicting the involvement of each functional entity through the various phases of the drug life cycle.

FIG. 3A is a diagram generally depicting the involvement of functional entities through the various phases of the drug life cycle and the structure of data management.

FIG. 3B is an additional diagram generally depicting the involvement of each functional entity through the various phases of the drug life cycle.

FIG. 4A is a diagram generally depicting an embodiment of a DLMS 100 in accordance with the present invention

FIG. 4B is a flowchart generally depicting the method of the present invention.

FIG. 4C-FIG. 4D are diagrams depicting further aspects of the DLMS 100.

FIG. 4E-FIG. 4F are diagrams generally depicting an implementation of a DLMS 100 in relation to various business entities of a life science organization.

FIG. 4G is another diagram generally depicting a possible are implementation of a DLMSU 90.

FIG. 4H is a flowchart generally depicting a method for collecting document data into a centralized data repository 125.

FIG. 4I is a diagram generally depicting the receipt of feedback concerning an a LSO productafter it has been released into the market place.

FIG. 4J is a flowchart generally depicting a method for collecting reports of deviations related to a drug product.

FIG. 4K is a diagram depicting one implementation of a status summary Dashboard.

FIG. 5 is a flowchart generally depicting a process for tracking and reporting unexpected events reported from the marketplace.

FIG. 6A is a flowchart generally depicting a process for receiving a request for testing and issuing a certificate of analysis.

FIG. 6B is a flowchart generally depicting a process for determining whether a particular piece of equipment is capable of conducting a specified test.

FIG. 6C is a flowchart generally depicting a process for determining whether personnel are capable of operating a particular piece of equipment to conduct a specified test.

FIG. 7A is a flowchart generally depicting a process for generating and distributing documents.

FIG. 7B is a diagram generally depicting an example of a report generated by the DMLS 100 based upon common rules and using data contained in centralized data repository 125.

FIG. 7C is a flowchart depicting a method of collecting drug data and initiating one or more responses in accordance with a common rules set.

FIG. 8A-FIG. 8C are diagrams depicting a possible implementation of a DLMS 100.

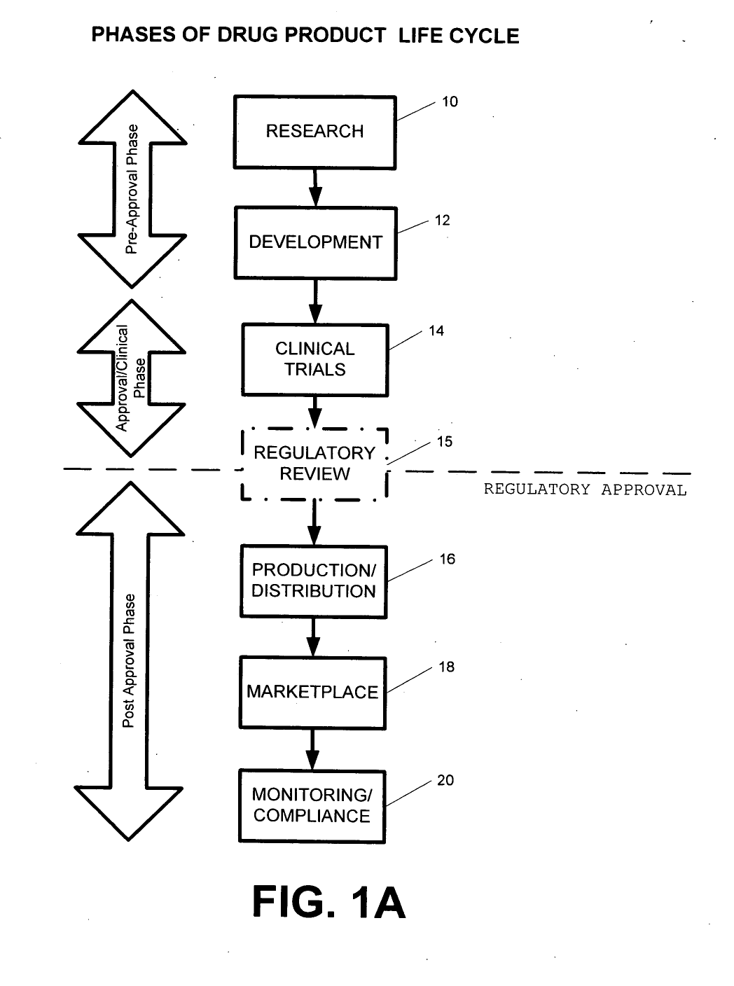

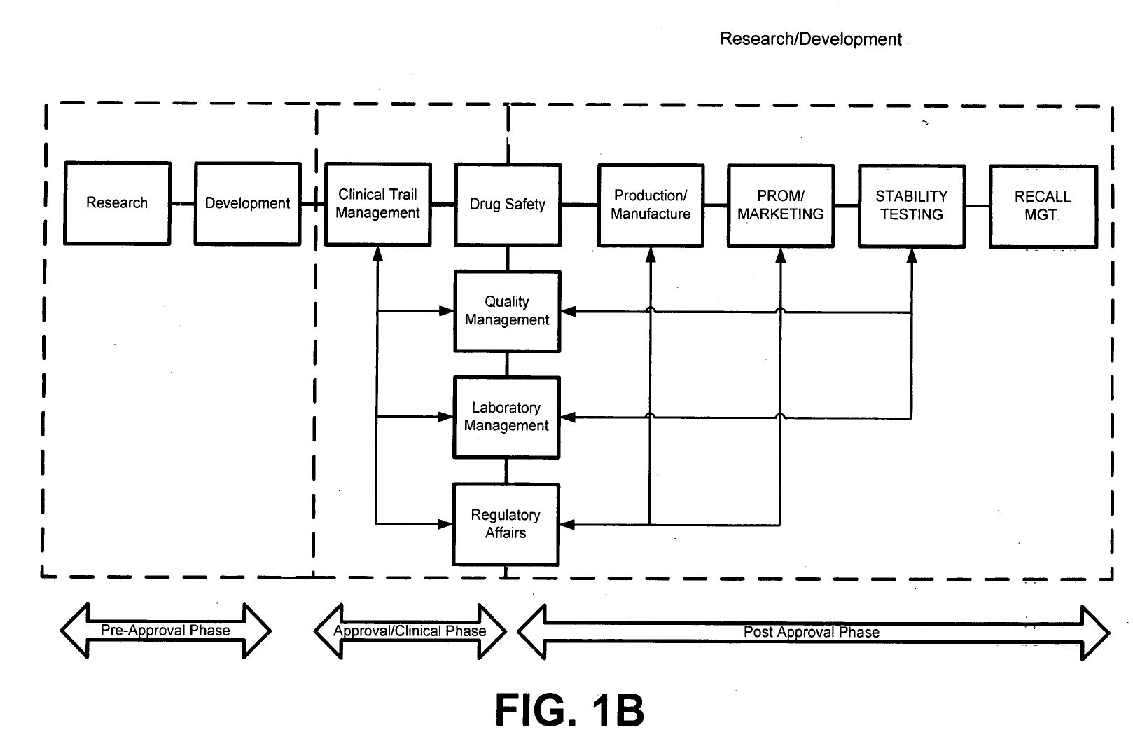

### DESCRIPTION OF THE INVENTION

The present invention is directed to providing a life science organization with tools for rules monitoring, notification management and trend analysis across the full spectrum of the life cycle of a LSO Product. The system provides a single solution to the complex and dangerous business of inventing, approving and ultimately having the LSO product enter the human or animal body either orally, transdermically, hyperdermically or surgically. The system provides a LSO the ability to proactively manage the LSO products and thereby mitigate the risks to consumers, and thus, reduce exposure to the consequences of running afoul of the many regulations and regulatory authorities world wide.

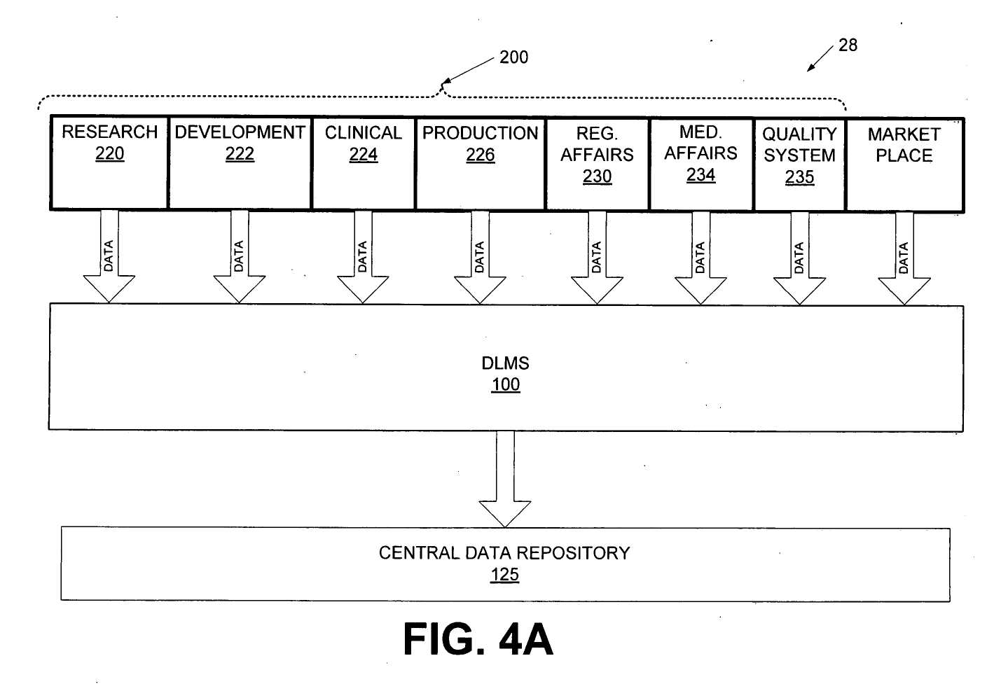

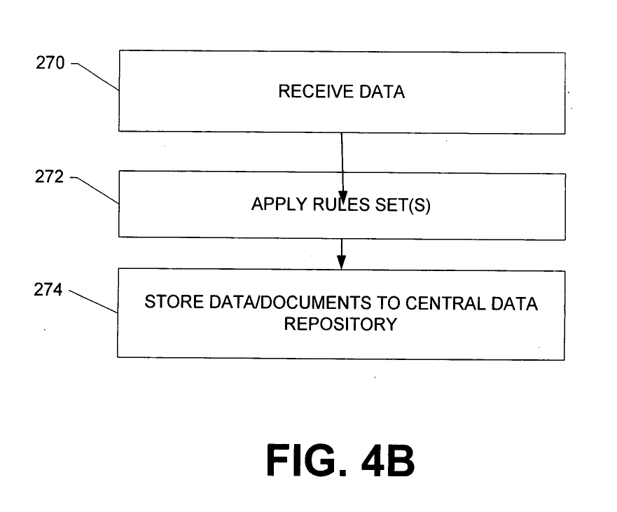

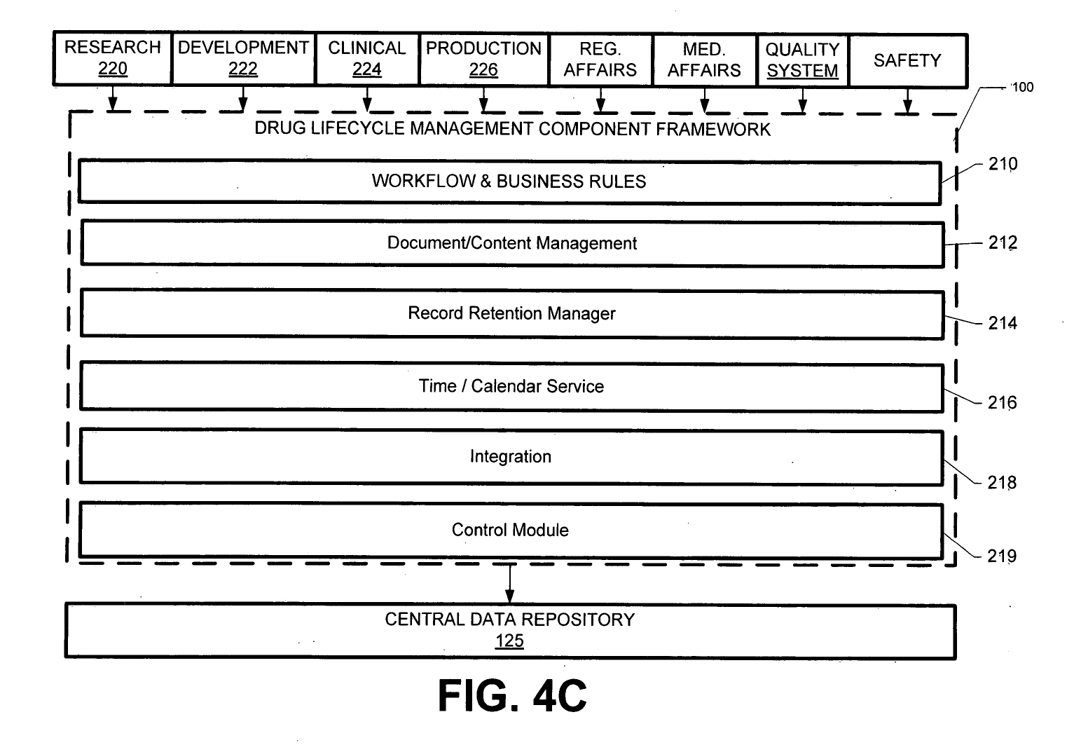

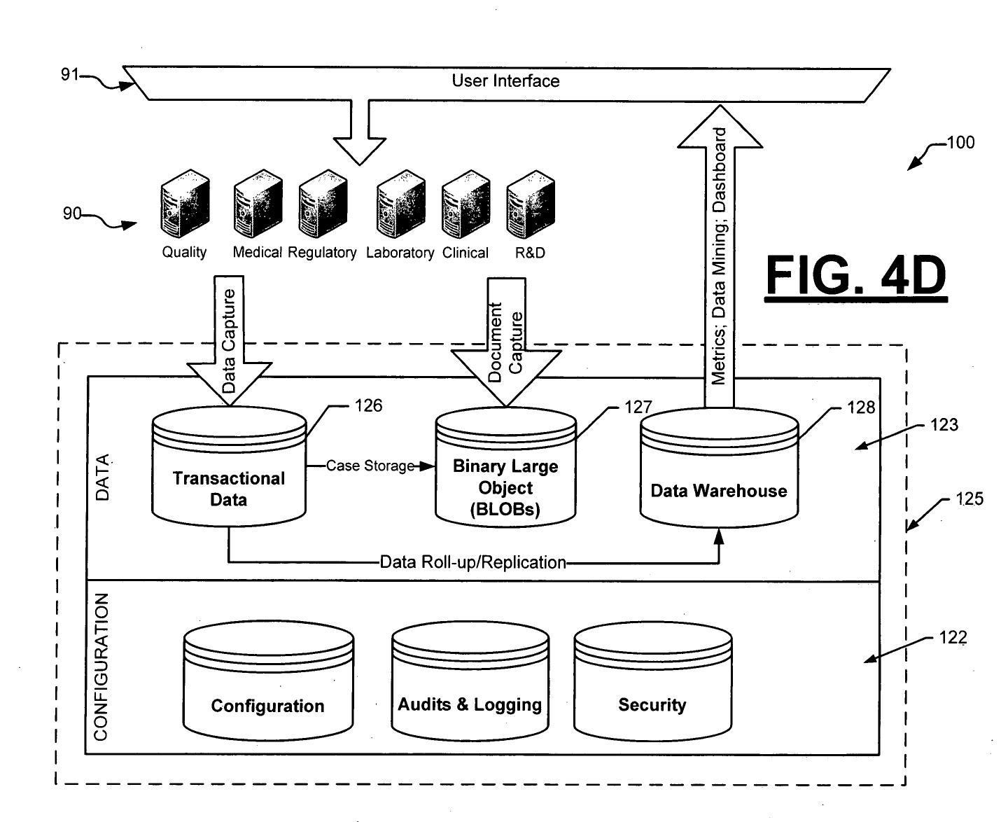

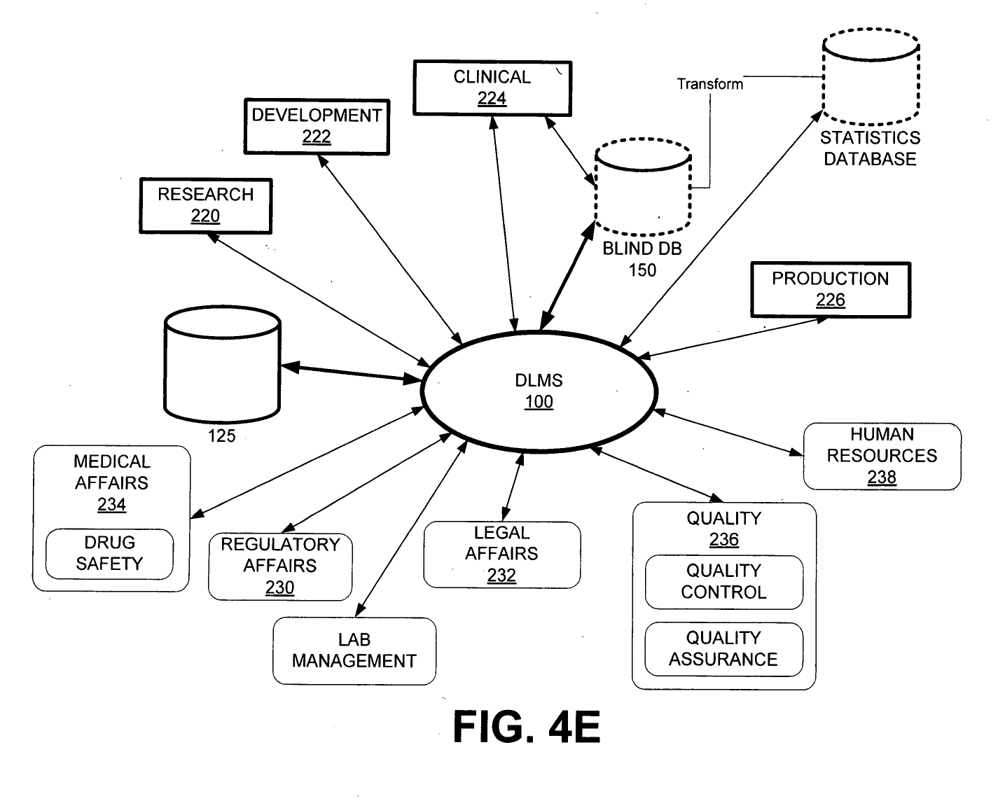

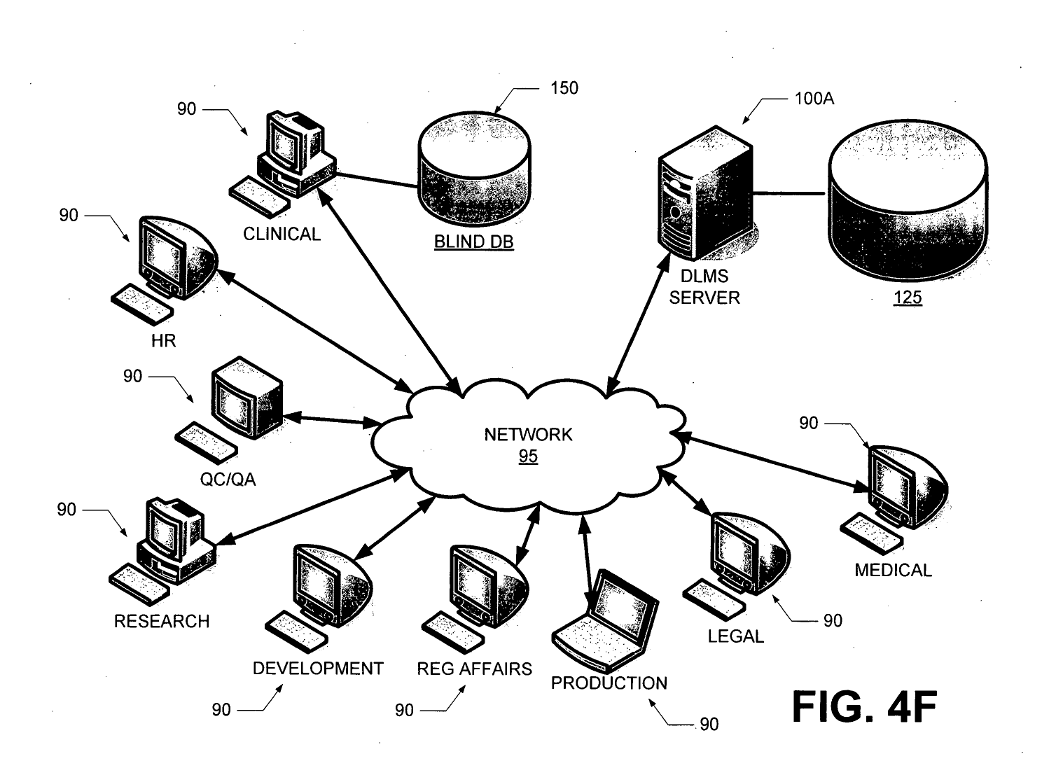

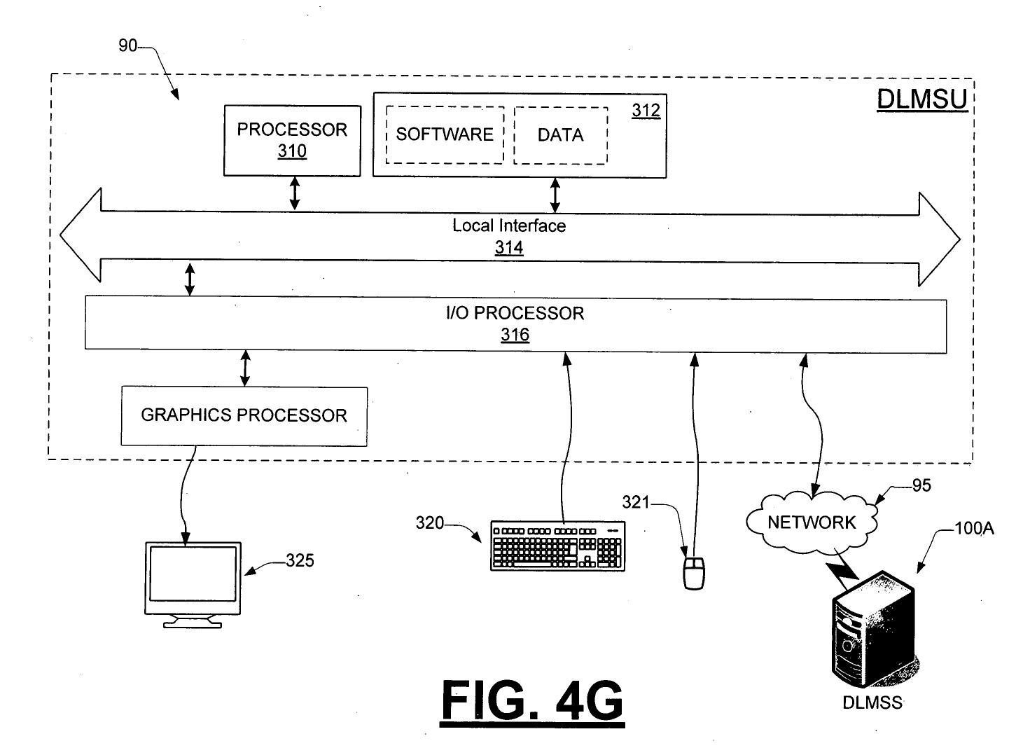

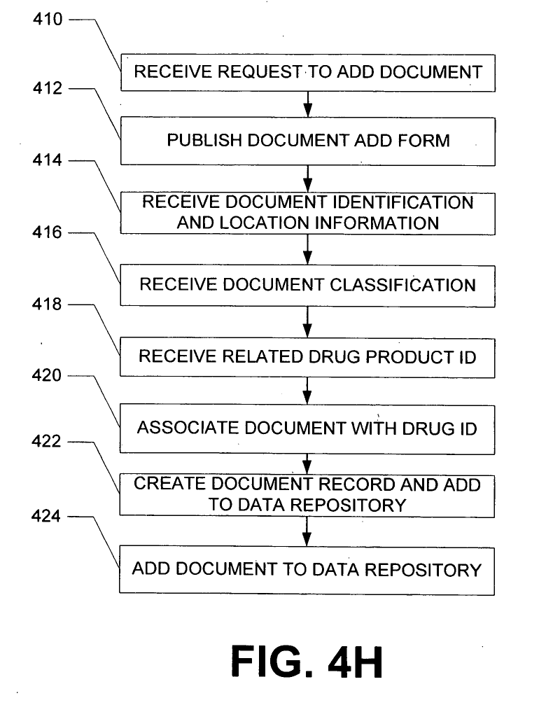

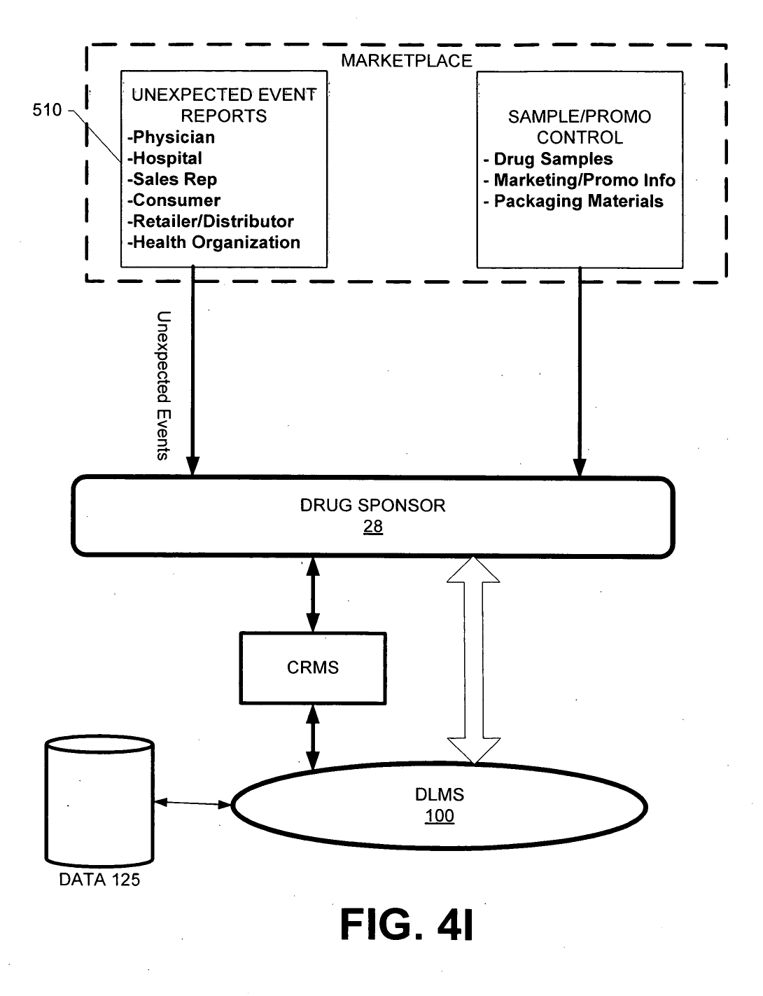

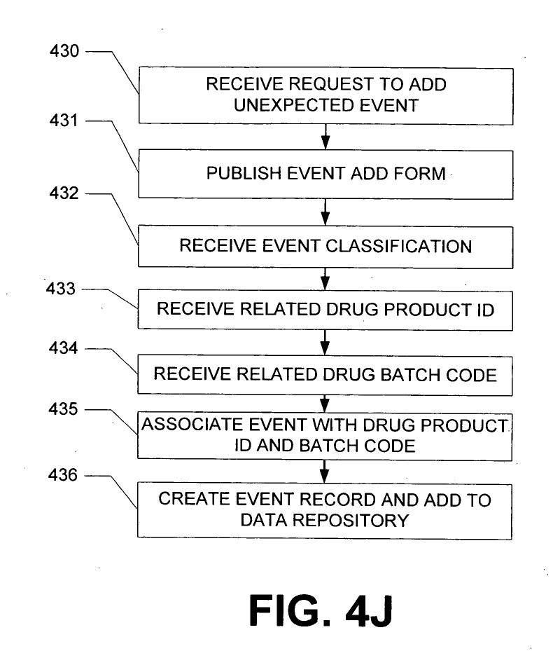

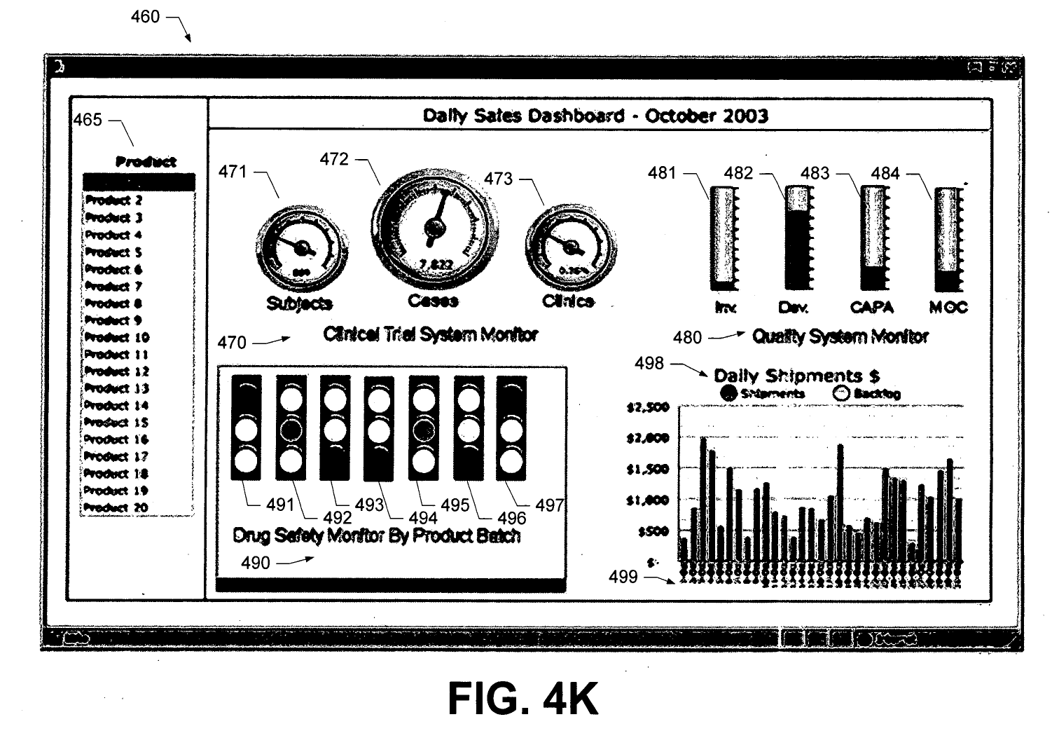

FIG. 4A is a diagram generally depicting a Drug Life Cycle Management System (DMLS.) of the present invention. The DMLS 100 is configured to receive data from each of the business entities 200 involved during the life cycle of a drug product. This data may include raw data, documents and/or files. Ultimately this data received from the various business entities is added to and stored in a central data repository 125.

Throughout the lifecycle of a LSO product, data is submitted to and received by the DLMS 100 from all business entities 200 that are involved with the LSO product as well as entities within the marketplace (consumers, physicians, hospitals, etc.). With reference to FIG. 4B, as data is received (270) by the DMLS 100 (FIG. 4A), it is subjected to a common set of predefined rules (272). In short, all data is subjected to the same standard no matter which business entity the data is received from. These rules define when certain predetermined actions are required for any given piece of data that is received. All data received from the various business entities is then stored into a central data repository 125 (274).

By subjecting all data received to the same standard of rules, it is possible for all potential issues and risks that a particular piece of data might raise to be addressed proactively. For example, if a report is received by the LSO that indicates that certain product information literature as typographical errors, more than one business entity may have an interest in knowing about the report and have separate and distinct responsibilities with regard to responding to the report. For example, the marketing department may need to know about the report so that the subject materials can be corrected, while the regulatory affairs Department may need to know about the report so that a required report of the typographical error can be made to relevant regulatory authorities.

Unfortunately, in a typical scenario, a report of the typographical error is received by someone associated with one of the business entities involved in the LSO who then determines that since it is marketing materials that are at issue, the matter should be forwarded to the marketing department for correction. No thought or recognition is given to the fact that such typographical errors and/or the correction thereof could also present regulatory, medical and quality compliance issues that should be dealt with by, for example, the relevant business entity. By subjecting all data to all predefined rules, the chances of an incident/issue/event related to a particular LSO product to “fall through the cracks” or otherwise not be fully addressed/responded to are greatly reduced, if not completely avoided.

One embodiment of the DLMS 100 is depicted in FIG. 4C. The DLMS 100 incorporates a workflow & business rules module 210, document & content management module 212, Record Retention Management Module 214, Time/Calendar Service Module 216 and integration server module 218. Each of the modules 210-218 are configured to provide service/functionality for each of the various departments/functional entities of the LSO 28.

Workflow & Business Rules Module 210 is configured to provide business process automation, through-out the integrated environment, with the ability to trigger processes, such as generation of notifications and/or alerts and document/report assembly processes as well as events across all departments and disciplines in the company. In addition it provides for the automation of many hundred of compliance and business rules such as deadlines, due dates, expiration dates and many others to ensure people and the company do not miss these important conditions.

Document & content management module 212 is configured to provide a central repository for all the company documentation from all departments that pertains to a particular drug product. These documents may be generated or collected by any one or more of the business entities involved in the drug life cycle, including, but not limited to Research, Clinical Trials, Quality; Regulatory; Medical; Legal departments. Documents may include SOPs; Investigation reports, customer complaints; batch test and analysis records; Adverse Event reports; regulatory authority reporting; CAPA; Change Controls; and manufacturing reports, to name a few.

Record Retention Management Module 214 is configured to provide rules associated with, for example, Federally mandated record retention periods. Among other things, these rules specify what period of time a particular document, or type of document, must be retained by the company. As there may be different time frames applicable to different document/types of document, there may be multiple predefined rules that may be applied for a given set of documents/information.

Time/Calendar Service Module 216 is configured to provide a centralized system for identifying where, at what time, and in what time zone work is being performed, and being time and date stamped—each time calculation is based on the GMT standard and converted to the appropriate time zone in which the work is being conducted. Integration server module 218 is configured to provide an interface for access to other computer systems; laboratory instruments/test equipment and external systems such as the FDA and service providers. A control module 219 is provided to coordinate and/or control the operations of one or more of the other modules.

The DLMS 100 provides a means to allow a LSO to proactively respond to conditions/circumstances that could signal potential risks/problems with LSO product already in the market place. In short, the DLMS 100 provides for monitoring transactional events that have occurred during the drug life cycle and, based upon predetermined thresholds or other criteria, determining whether or not the body of transactional events (transactional history) indicates that a problem may exist or that certain action may be/is required. The DLMS 100 may then cause an alert or notification to be generated and forwarded to specified personnel/business entities. The DLMS 100 may also generate and provide a checklist (response action plan), of action that may be required to address a particular situation, to be included in or linked to, a notification or alert.

The DLMS 100 is configured to collect data relevant to a specific drug beginning with the research and development effort, through clinical trials phase, to production/manufacturing of the drug to distribution of the drug into the marketplace. This data is collected into a centralized data repository 125 that is associated with the DLMS 100.

In one embodiment, the data repository is composed of one or more transactional databases, one or more binary large object (BLOB) databases and one or more data warehouse databases, as well as one or more secure configuration databases for storing data related to system configuration, security and auditing and logging.

The DLMS 100 also provides for generation of reports and/or applications necessary for compliance with regulatory requirements.

FIG. 4D depicts one implementation of DLMS 100 in which central data repository 125 includes system related configuration database(s) 122 as well as data storage databases 123. The configuration databases 122 store information related to the DLMS system configuration, security, as well as auditing and logging information concerning system changes and usage. As data is collected a record reflecting the data is entered into a transactional database 126. Any where the added data is a document and/or file, such data is stored into binary large object (BLOB) database 127 and a link to the document is created in the corresponding data record in the transactional database 126. Data stored in the transactional database 126 is replicated in a data warehouse 128. This data warehouse 128 is monitored and various metric and/or analytical processes may be carried out based upon the data stored in the data warehouse 128. In one embodiment, the a summary dash board may be provided that provides a snap shot of the current business conditions, based upon the data stored in data warehouse 127.

The centralized data repository 125 may be implemented as a single database or, alternatively, as a set of databases. The centralized data repository 125 may be distributed across one or more locations or servers. The centralized data repository 125 may be composed of one or more databases. These databases may be distributed across a network and/or located across multiple servers or physical locations. Additionally, the databases may be any one of a number of types of database including, for example, relational databases, binary object (BLOB) database, or a data warehouse database, or any combination thereof.

With reference to FIG. 4E each of the various business entities 200 (departments/organizations 220, 222, 224, 226, 230, 232, 234, 236, etc.) associated with the LSO 28 generate data related to a LSO product and/or retrieve or access data related to a LSO product for review or document/report generation or other purposes. This data is collected by the DLMS 100 and may include documents generated by the business entity in connection with a drug product, such as, for example, marketing and/or promotional materials, LSO product packaging materials, and reports or statements generated in accordance with regulatory requirements. Other data that may be collected includes reports and information received from third parties in connection a LSO product after it has been released into the marketplace. This data may include, for example, documents as well as information connected with reports of unexpected events that occurred or allegedly occurred in connection with a particular drug product. Further, data collected may include internally generated information such as corrective action/preventive action related documents prepared in the course of an investigation of, for example, a reported unexpected event, as well as whether or not a case or investigation has been opened or closed.

With reference to FIG. 4F a DLMS unit 90 (DLMSU) may be associated with each business entity 200. For example, for each of the business entities 220, 222, 224, 226, 230, 232, 234, 236, etc. an associated DLMSU 90 may be respectively provided. Each of the DLMSU 90 is preferably configured to access a network 95. A DLMS server 100A may be provided and is -connected to the network 95. Communications between the DLMSU 90 and the DLMS server (DLMSS) 100A may be carried out over the network 95. The network 95 may be a local area network or a wide area network, such as the Internet, or a combination of both. Network connections may be implemented via wired or wireless means, however may be desired or available.

FIG. 4G is a diagram depicting a possible implementation of a DLMSU 90. In this example, the DLMSU 90 is configured to include a processor 310 and memory 312, for storing software and data. A local interface 314 is provided to allow the processor 310 and other components of the DLMSU 90 to exchange instructions and/or data. An I/O processor 316 is provided to receive input from input devices such as keyboard 320 and pointing device 321. It also provides for output of data to graphics processor 318 for generation of an output video or graphics signal to display device 325. The DMLSU 90 is preferably configured to run a web browser application, such as, for example, Microsoft Internet Explore, Mozilla FireFox or Opera. Software 1311 may be configured to carry out any one or more of the functions and processes described herein and/or shown in the flowcharts herein, including that of web browser functions.

Clinical Trials-Blind Database

In order to avoid the possibility of interested parties attempting to influence the outcome of ongoing clinical trials, the DLMS 100 is configured to receive the clinical trials data and collect it into a blind database which is shielded from access by, or disclosure to, all research parties for the duration of the clinical trials. By shielding clinical data from access outside clinical personnel during the pendency of the clinical trials, it is very difficult, if not impossible, for anyone to sway or otherwise influence the outcome of the clinical trials.

DLMSU 90 used by clinical department 222 is associated with a blind database 150. This blind database 150 is provided to collect clinical trials data generated during the clinical trials operations, typically conduct and/or overseen by the clinical trials department 24. The blind database 150 is separate and distinct from the centralized data repository 125 and is provided to maintain a barrier to access of the trials data by anyone not involved with conducting the clinical trials. In a preferred embodiment, the blind database 150 is not accessible to anyone other than those departments/personnel involved in the conduct of clinical trials. Once clinical trials are complete, all trials data is transformed or transferred to a Statistical Database for analysis, while keeping the Clinical data collected in the blind database untouched and “clean” so that it can be used as a reference database. This may be accomplished in accordance with the process described and discussed hereafter with respect to the flowchart of FIG. 4H.

By providing a blind database 150 during clinical trials, the chances of parties attempting to make changes to the trials protocol in order to influence the outcome of the clinical trails is greatly reduced, if not eliminated.

Collecting Data-Documents

In one embodiment, the DLMS 100 is configured to collect or otherwise receive data from each of the various business entities (departments/organizations) within or associated with the drug sponsor 28 throughout the life cycle of a drug product.

In, for example, the case of the research department 232, research notes, experimental data and results may comprise a large portion of the research data generated by the research department. These research notes, experimental data and results are typically generated by research personnel in various formats, including hard copies of text, spreadsheets, graphics, photographs, etc., as well as electronic formats, such as, for example, word processor files, spreadsheet files, hyper-text mark-up language (html), image or multimedia files, universal resource locator (URL) links to internet web sites or resources on a local area network, and others.

As an example of one implementation of the present invention, the case of research data generated by, for example, the research department 220 will be discussed. In this example, a department repository file directory (Research Database) is established for storing all data related to a particular LSO product or product identifier (product ID). This data may be generated directly by personnel or via automated testing and monitoring equipment that outputs research data.

Where research data is embodied in a non-electronic format or hard copy, such data must be converted into an electronic format before can be is placed into the research database.

With reference to FIG. 4H, the DLMS 100 may be configured to allow personnel to add documents or data files to the DLMS 100 data repository 125. One example of the process followed by the DLMS 100 in adding a document to the data repository 125 is generally depicted in FIG. 4H. Once a request is received (410) to add a document to the data repository 125, the DLMS 100 causes a ADD DOCUMENT form to be published by the DLMSU 90 that has requested to add a document (412). The form is then completed by the requested party and submitted. The DLMS 100 then receives document identification and location information that was provided by the form (414). Document classification is received (416) as well as the product code for the drug that the document is related to (418). The document is then associated with the LSO product code/ID (420) and a document record is created and added to the data repository (422). The document is added to the data repository (424)

Collecting Data-Unexpected Events

Reports of unexpected events related to a LSO product is vital in avoiding or limiting the potential for harm to consumers due to a LSO product or the drug in combination with other drugs; illnesses, organ maladies and event the use of alcohol or illegal narcotics. The DLMS 100 is configured to receive reports of unexpected events and to create records of each reported event in the central data repository 125. FIG. 4I is a diagram generally depicting the manner in which adverse events are reported to the drug sponsor (LSO) once a drug has reached the marketplace. These reports of adverse events may come from multiple sources 510, including, but not limited to physicians, hospitals, LSO product sales representatives, consumers, retailers/distributors and health organizations. The process of collecting event reports is generally depicted in the flowchart of FIG. 4J.

With reference to FIG. 4J, the DLMS 100 receives a request to add an unexpected event report (430). The DLMS 100 then causes an event add form to be generated and published on a DLMSU 90 that is associated with the party requesting to add an event report. The form is then filled in and the data is submitted to, and subsequently received by the DLMS 100. A classification for the reported event is received (432). A related LSO product ID is received (433), as is the related drug batch code, if any (434). The reported event is then associated with the LSO product code and the batch code, if any (435). A record of the event is then created and added to the data repository 125 (436).

In one embodiment, the DLSM 100 is configured to add a record reflecting data/document data to the central repository 125 into the transactional database 126. The transactional database 126 may be configured as a relational database. The data/records in the transactional database 126 is replicated in the data warehouse 128 (FIG. 4D) for monitoring (constant monitoring, for example) trending and easy analysis of the data without interfering with continued collection of data into the transactional database 126.

Keeping Track of the Big Picture—Product Status Summary Dashboard—Given the complexity of the process involved in bringing a LSO product to market, one implementation of the DLMS 100 is directed to providing relevant management/executive personnel with a quick and simple set of monitors that indicate the current status of various aspects of a drug product. Each dashboard is implemented to provide any one of a number of “views” into the data, from any perspective such as product; event or deviation. With reference to FIG. 4K a sample scenario is illustrated in which a summary dashboard is provided. One embodiment the DLMS 100 may be configured to generate and publish a dashboard summary 460 for a given drug product. In this example, the dashboard 460 is published as a daily sales dashboard. It provides several monitors 470, 480, 490 and 498. These monitors incorporate various graphical indicators 471, 472, 473, 481, 482, 483, 484, 491, 492, 493, 494, 495, 496 , 497 and 499 that describe the current status of various aspects related to a selected LSO product 465 (in this example, product 1). The data on which the dashboard indicators are based is contained in data warehouse 128 (FIG. 4D).

A clinical trial system monitor 470 is provided that includes indicators 471-473. Indicator 471 shows how many subjects are/were enrolled in clinical trials. Indicator 472 shows how many trials cases are currently open. Each dashboard can be configured to display information related to the specific user and/or their responsibilities. Indicator 473 is provided to show how many clinics are involved in the clinical trials.

A quality system monitor 480 is provided that includes indicators 481-484. Indicator 481 shows how many Investigations have been opened or are in progress. Indicator 482 shows how many Deviations have occurred or been reported to the Quality Department. Indicator 483 shows how many Corrective Actions/Preventive Actions (CAPA) have been initiated, implemented and closed. A product shipment monitor 498 is provided that includes indicators 499, each of which corresponds to a particular date or time frame and indicates the volume of product shipments and/or product shipment backlogs.

Drug Safety Monitor 490 is provided that includes indicators 491-497. Each of the indicators 491-497 correspond to a particular batch number of the selected LSO product 465. These indicators are configured to provide: a GREEN light when no unexpected events have been reported in connection with a particular batch; a YELLOW light when the level of unexpected events reported in connection with a particular batch is within a predetermined range and a RED light when fatal events are reported. The status represented and indicated by each of the indicators of the various monitors 470, 480, 490 and 498 is based upon relevant event data contained in the centralized data repository 125. This green light, Red light configuration, as well as any of the other monitor configurations may be provided to indicate any variable/factor concerning a LSO product that is of interest.

Feedback from the Marketplace-Adverse Events—As discussed above with respect to FIG. 4J once a LSO product reaches the marketplace, the LSO 28 can receive reports of adverse events related to a particular drug product. These adverse events may be categorized as medical adverse events (AE) or technical complaints. A medical adverse event (AE), may be, for example, a chemical interaction of the LSO product in the patient. While a technical complaint relates to, for example, a failure of some kind in the product, such as crushed tablets in the packaging, or disintegration of the LSO product when exposed to direct sunlight or high levels of moisture. Under certain circumstances a Technical Complaint can result in an AE. For example a patient might take an over-dose of the medication because it had disintegrated in its packaging and the patient did not know the exact amount of the resultant powder to consume.

In the case of AEs, which can be classified as into several types including: expected and non-serious; unexpected and non-serious; unexpected and serious; expected and serious or fatal, the type of AE determines the severity or level of the responsive action required by the LSO. Additional factors may be considered in determining what responsive action will be required/taken including the reliability of the source of the AE. For example an AE received from a hospital or a Physician receives a higher level of credibility than does that of a consumer, this is due to the level of knowledge of the patient and understanding of their medical condition and any other medications they may be taking, information that helps determine the root cause of the AE.

The LSOs response in the event of a fatality requires immediate action on the part of the LSO. The DLMS 100 is preferably configured to immediately notify all affected parties of the AE and sets in motion a process for meeting the reporting requirements imposed by regulating authorities.

In the event of an expected non-serious AE, for example the LSO product may cause headaches, the DLMS 100 is configured to store this information for inclusion into, for example, a Period Safety Update Reports (PSUR) required by regulatory authorities. It is further configured to automatically determine the length of time the LSO product has been approved for sale and to flag the a relevant record to be included in the quarterly PSUR report, if the drug has been approved for example, for three years or less and if longer to have it included in an annual PSUR report.

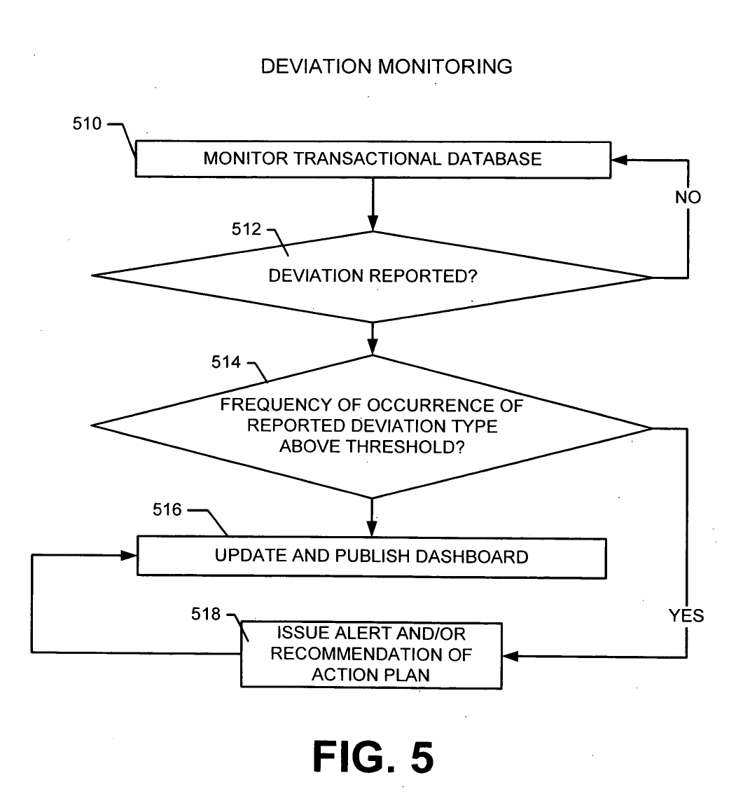

Technical Complaints and Laboratory Deviations—FIG. 5 is a flowchart depicting a possible implementation of the process of monitoring deviations recorded by the DLMS 100 to the central repository 125. If a deviation, whether it takes the form of a Technical Complaint, OoS, laboratory deviation, or Out of Trend (OoT), is reported (512) a determination may be made as to whether or not the frequency of the occurrence of a type of deviation has exceeded a predetermined threshold (514). If not summary dashboard indicators can be updated to reflect the reported deviation (516). If the frequency of deviation reports does exceed the predetermined threshold, an alert notification is issued and forwarded to one or more predetermined parties (518), for example, to either or both departments/parties responsible for drug safety and/or quality control. This notification may also include a recommended or required set of steps (action plan checklist) that should/must be taken to address the situation and/or otherwise ensure compliance with regulatory requirements related to the occurrence of deviations or certain types of deviations. This notification may be issued via e-mail and include one or more links to all data contained in the central repository 125 that pertains to the reported deviation. This data will preferably be composed of all data that will be necessary to investigate the deviation and establish a corrective action/preventive (CAPA) action plan. For example a link directly to a listing of records contained in the central data repository 125 for recent deviations of a particular type could be included in the e-mail message. Alternatively, alert notifications may also be issued via telephone or text mail or any number of other communications mediums. The dashboard may then be updated to reflect the above threshold status of reported deviations (516).

In one embodiment, if an response action plan/checklist is provided as a part of, for example, an alert notification, the DLMS 100 (will generate and calendar follow-up times and/or dates. Subsequently, on the calendared follow-up dates the DLMS 100 will cause a status inquiry notification to be generated and forwarded to the responsible party/parties to elicit input as to the status of the items set out by the action plan checklist.

Corrective Action/Preventive Action—Once a CAPA corrective action plan has been designed/established by personnel investigating a reported deviation, it will be submitted to the DLMS 100 for incorporation into the central data repository 125. The DLMS 100 is configured to establish and store a schedule of tickler dates for each step of the CAPA action plan based upon the submitted CAPA action plan. Subsequently, as each tickler date arrives, the DLMS 100 is configured to issue a status inquiry notification to parties responsible for carrying out the CAPA action plan. This status inquiry will preferably be issued by e-mail to a predetermined party/e-mail address and may include, for example, a link to a status update form published by the DLMS 100. Upon receiving the status inquiry, personnel may go to the status inquiry update form via clicking/activating the included link. Once at the published status form, personnel may indicate the status of the step by choosing, for example, one or more check box options, for example: COMPLETED; IN PROGRESS or UNABLE TO COMPLETE.

Testing and Analysis-Issuance of Certificate of Analysis—In order to reduce the occurrence of problems associated with flawed testing and analysis of drug components, due to improperly trained personnel or un-calibrated test equipment being utilized in conducting a test, one embodiment of the DLMS 100 is configured to allow a test to take place, and subsequently a CofA to be issued, only when central data repository 125 indicates that 1) the test equipment used to conduct the test was properly calibrated for the entire duration of the test and 2) the personnel operating the test equipment was properly trained on the equipment and was also current with continuing professional education (CPE) requirements. By pre-checking personnel training and equipment calibration records, poorly or untrained personal are excluded by the DMLS 100 from being selected to run tests. In addition no instruments that fall out of calibration before or during the tests may be used. The DLMS 100 is also designed to ensure that any other test criteria are also adhered to, and, in the event of a failure of any part of the test due, automated Deviation is noted/recorded to the centralized database and a notification is generated and forwarded appropriate personnel.

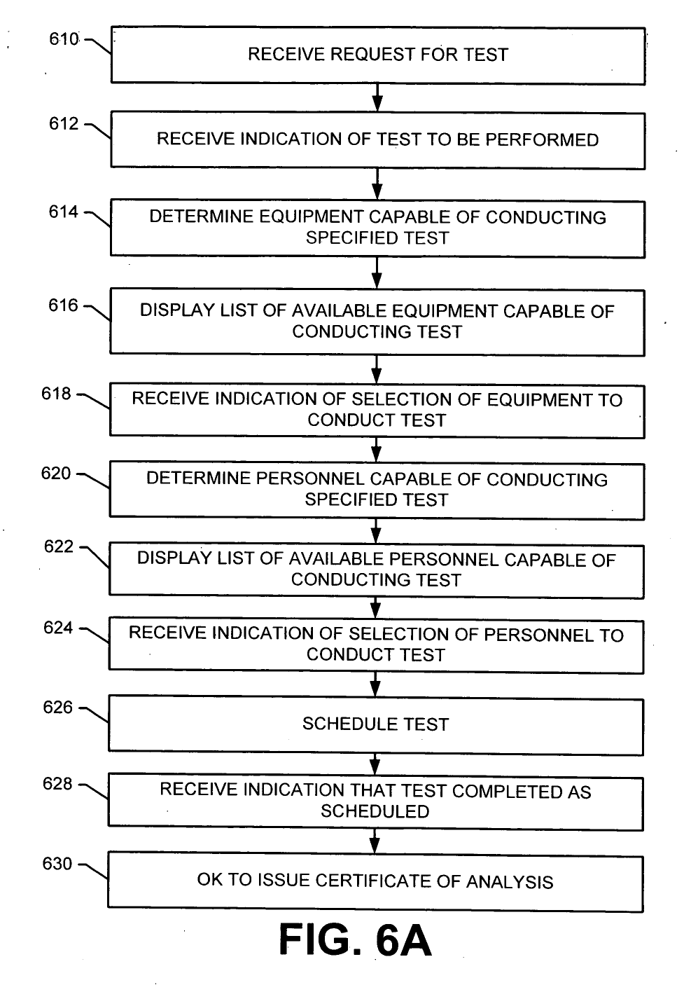

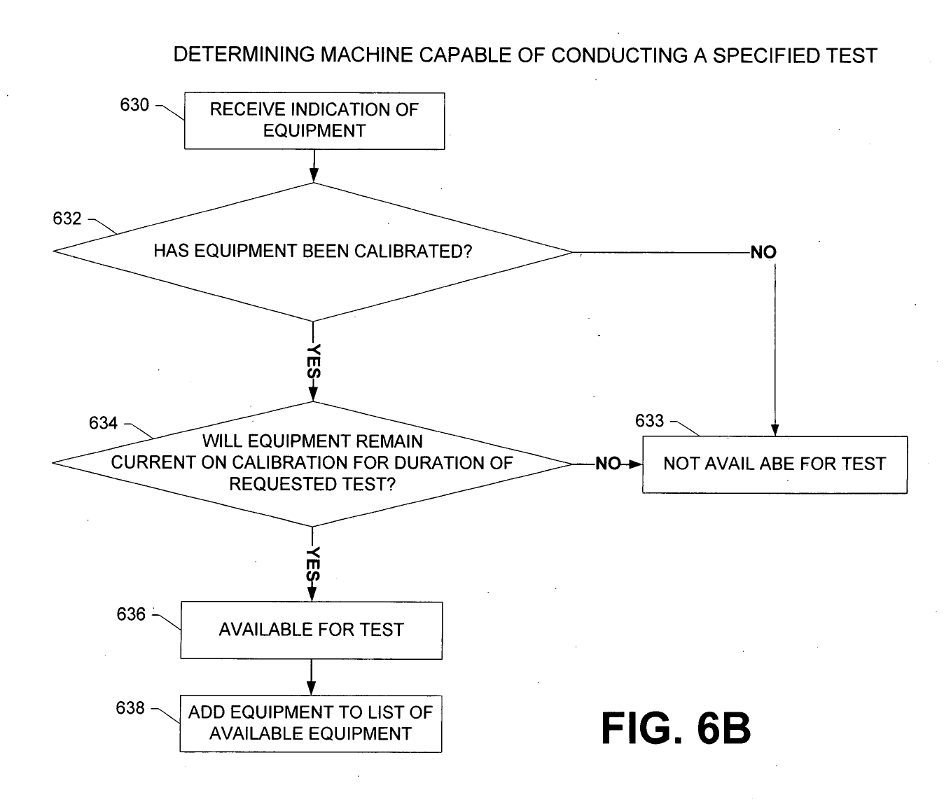

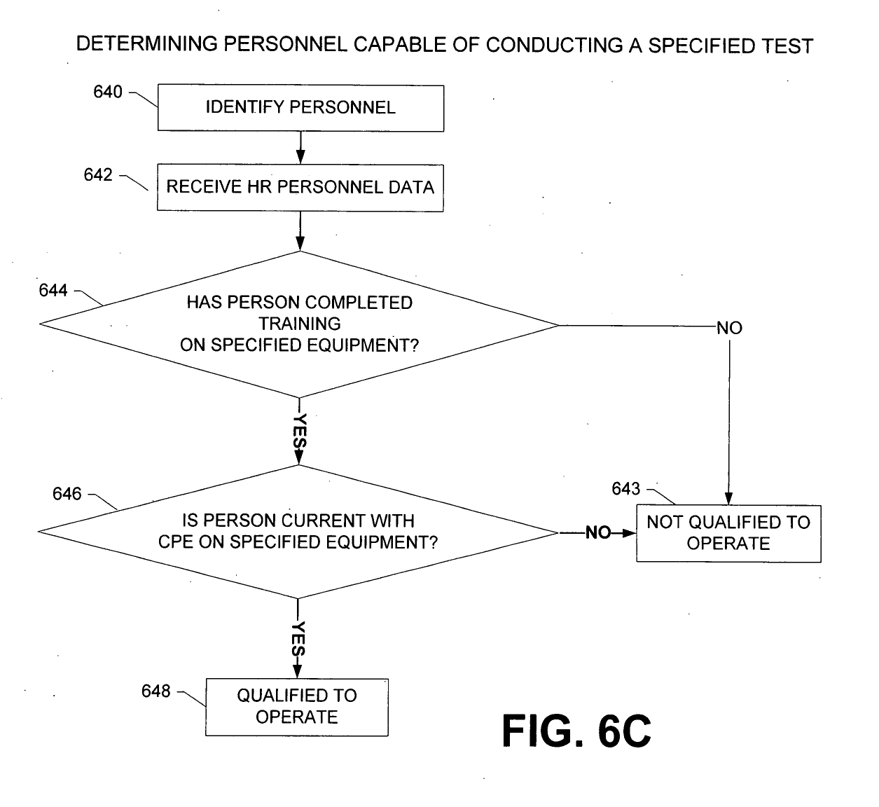

FIGS. 6A-6C are flowcharts generally describing the process of issuing a certificate of analysis performed by one embodiment of the DMLS 100. With reference to FIG. 6A, the DMLS 100 is configured to receive a request for a test (610). An indication of the type of test to be performed is received (612). A determination is then made of what equipment is capable and available to carry out the specified test (614). Once this determination is made, a list of available equipment is generated and published for display (616). An indication of which equipment is selected is received by the DMLS (618). This indication may be provided by a user making a selection of the particular equipment via highlighting the equipment on the display device on which the equipment list is displayed. A determination is then made of personnel who are capable of carrying out the specified test (620). A list of available capable personnel is then generated and published for display (622). An indication of which equipment is selected is received by the DMLS (624). The test is then scheduled for the selected equipment and personnel at a specified time (626). Once an indication is received from authorized personnel that the test has been successfully completed as scheduled, an approval to issue a certificate of analysis is issued (630). Alternatively, the certificate of analysis may be automatically generated and submitted to appropriate parties for approval, if needed.

To determine what equipment is capable of carrying out a particular test, the DLMS 100 is configured to prevent any equipment that is not properly calibrated or capable of remaining calibrated for the duration of the requested test. Since certain test may take many hours to complete, it is possible to begin a test while the equipment is still current with calibration, only to have it fall out of current calibration status at some point during the test. Although the test is complete, the results can not be guaranteed to be accurate and they should not be relied upon. If for some reason equipment should fall out of calibration and is either manually detected or provided via an automatic signal from the equipment, then a deviation alert is triggered in accordance with, for example, predetermined quality rules and an investigation is initiated. The result is that the test must be run again on equipment capable of remaining calibrated for the duration of the test.

FIG. 6B is a flowchart generally depicting a process for determining what equipment is capable of conducting a requested test (as specified by the step 614 of FIG. 6A). With reference to FIG. 6B an indication of equipment type or model of equipment is needed is received (630). The DLMS 100 then determines if the equipment has been calibrated (632). If not, then the equipment is deemed not available for testing (633). If so, then a determination is made as to whether or not the equipment will remain current on calibration for the entire duration of the requested test (634). If so, the equipment is deemed available for the test (636) and is added to the list of available equipment (638). Otherwise, the equipment is deemed not available for test (633).

FIG. 6C is a flowchart generally depicting a process for determining what personnel are capable of conducting a requested test on a particular piece of equipment (as specified by the step 620 of FIG. 6A). With reference to FIG. 6C once a piece of equipment has been determined to be available for a given test, personnel are identified (640). The DLMS 100 then determines if personnel has been trained on the particular equipment (644). If not, then the personnel is deemed not qualified to run test (643). If so, then a determination is made as to whether or not the personnel is current with relevant CPE requirements (646). If so, the personnel is determined to be qualified to run the requested test as scheduled (648). Otherwise, requirement the personnel is deemed not qualified to run test (643).

Generating and Retaining Documentation-Controlled Documents—The DLMS 100 is configured to provide as a part fo the central data repository an Official Document Library (ODL). The ODL is provided to store any and all documents that are subject to regulatory audit or scrutiny.

Controlled documents may include, for example, but are not limited to, Quality; Regulatory; Medical; Legal departments and the Documents include SOPs; Investigations, customer complaints; batch records; Adverse Events; regulatory authority reporting; CAPA; Change Controls; manufacturing reports to name a few. All other documents are stored in their relevant sites such as R&D or Clinical. The controlled documents go through a formal Author; Review; Approval and electronic/digital Signature process before being classified and added to the Official Document Library (ODL) (not shown). Non-controlled documents are stored according to company policies or in the case of Clinical trials related document, they may be stored in a formal electronic Common Technical Document (eCTD) structure/format for easing drug related filings with regulatory authorities, such as, for example, the FDA.

Before being submitted to the DLMS 100 for incorporation into the ODL, a controlled document must be subjected to a peer review process. Once the peer review is completed, it is submitted to all required personnel for approval. Once all members of the process have approved the document it may be converted into a predetermined standard format, such as, for example a portable document file (PDF) format. This conversion may be initiated by, for example, the document control department (person). At this time a watermark is added to the document to embed a clear mark/insignia indicative of the fact that the document is a controlled document.

The document is then distributed to relevant parties for signature via electronic mail. Once all signatures have been applied to the document it is returned to, for example, the document control department (person) who then sets the document for classification in the ODL. At this point date for the next review of this document may be set and calendared for action. The document can then be placed into “production” for use by all eligible employees.

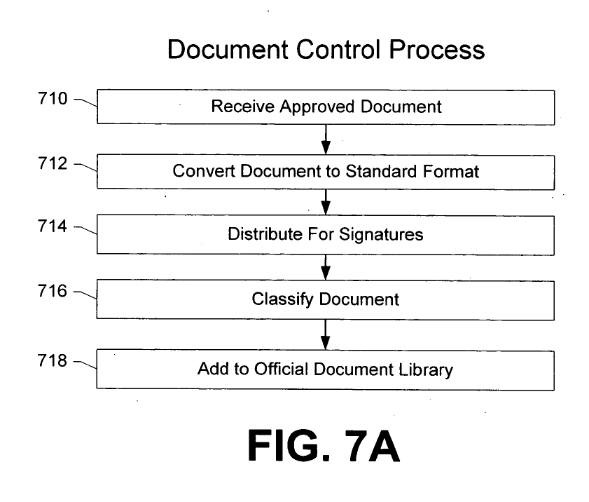

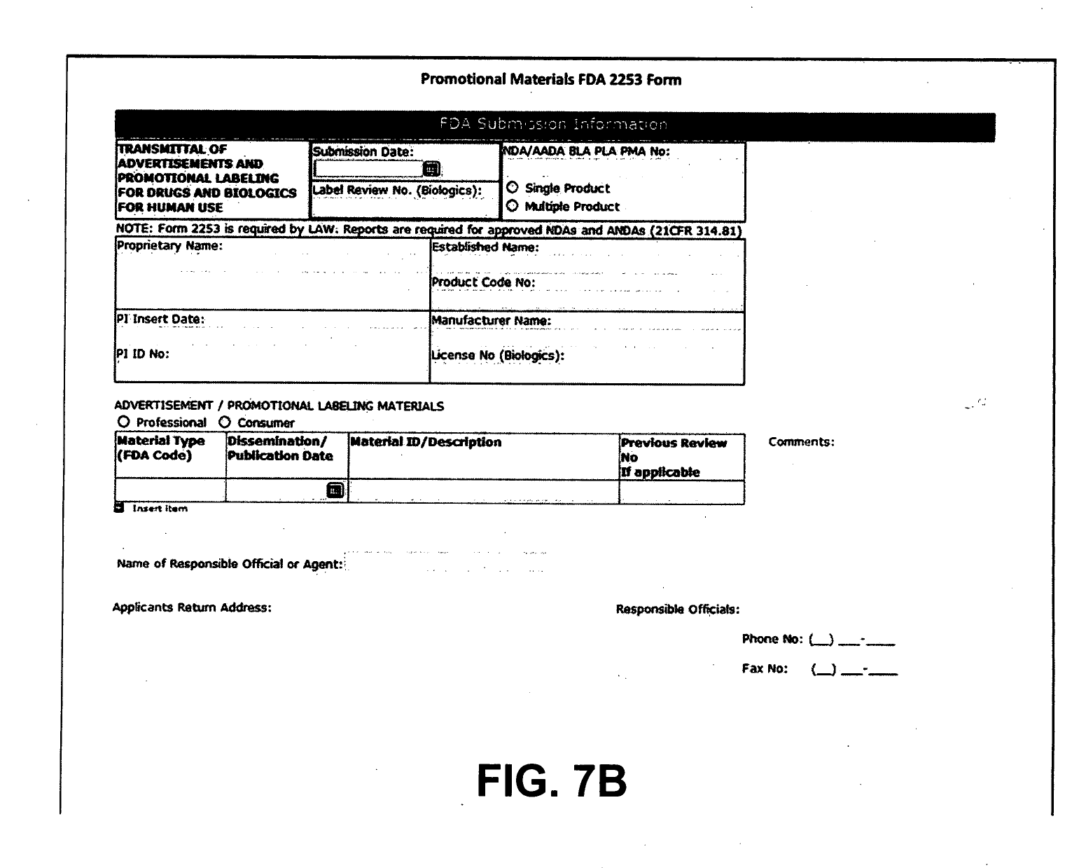

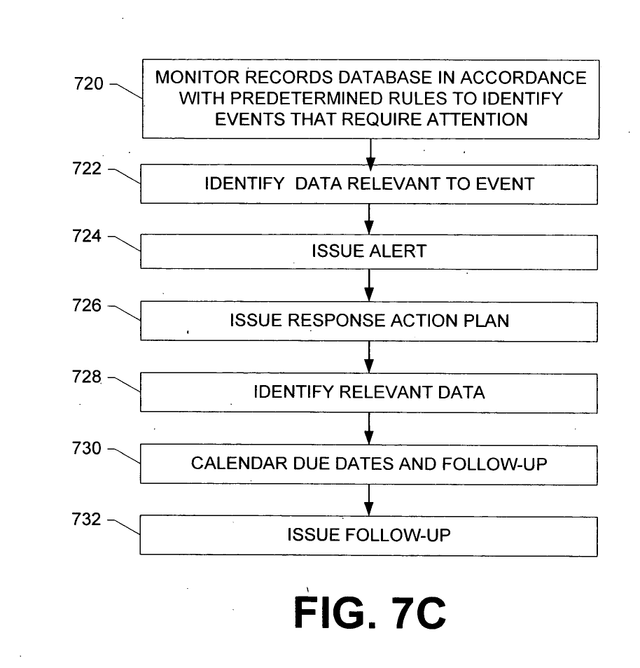

At every point in the process the user of the DLMS 100 is notified via, for example, email notification of their role and the duties they must perform at a particular point in the process. For example, if they are part of the document review group, they may receive an email asking them to review the document. The e-mail message may include a link (hyper-link) to the actual document. Additionally, the summary dashboard may be updated to reflect the in-process work that must be still be performed by the user or department responsible for the document review. One possible implementation of the process is depicted in the flowchart of FIG. 7A, wherein a document that has been approved is received by the DLMS 100 (710). The document is then converted into a predetermined common electronic format (712). The converted document is then circulated for signatures by appropriate persons (714). This document may be circulated by issuing a notification via e-mail that includes a link (hyper-link) to the document and/or a “signature” form that personnel must complete in connection with the document presented for signature. Once signatures are obtained, the document may be classified (716) and added to the ODL.

In a preferred embodiment, the workflow and business rules module 210 (FIG. 4) of the DLMS 100 includes a rules engine (not shown) that is configured to monitor the central data repository 125, specifically transactional database 126, to determine if any event records have been created/added to the central repository that act to trigger certain predetermined rules (business rules). These business rules define certain events/occurrences that should be regarded by the DLMS 100 as a trigger for further action(s) defined by the business rules.

Multiple types of business rules may be provided, including regulatory rules that specify such things as reporting requirements of a regulatory authority, such as the FDA; required responses and/or follow-up actions upon detection of a event record related to the occurrence of a particular event/occurrence/action or type of event/occurrence or action. Regulatory rules may be stored as a part of the data in the central data repository 125. Other types of business rules may include: 1) data retention rules that specify and control the period of time certain types of data are retained and/or when they are destroyed. These rules may be used to calendar data deletion dates for action; or 2) HIPAA (Health Insurance Portability and Accountability Act of 1996) compliance rules that insure that certain confidential information that may be submitted to for inclusion in the central data repository 125 of the DLMS 100 is properly masked or otherwise redacted/deleted so as to maintain confidentiality of, for example, trials subjects or consumers/patients of a LSO product when data is accessed or incorporated into other documents, such as required regulatory reports; or 3) Controlled Document Review and Approval Rules that set out the review, approval and signatory requirements for documents to be added to the Official Document Library (ODL).

In typical use, the DLMS 100 receives a request via a DLMSU 90 to add data to the central data repository 125. Upon receipt of the request, the DLMS 100 generates and publishes an appropriate document and/or or event data add form to elicit certain predefined information from the party requesting to add document/event data. The data add form may be designed to provide various form fields to elicit specific pieces of information such as, for example, the LSO product name or product ID or batch number of particular LSO product that the data is related to and/or. The form will elicit information that is relevant and /or needed. The information elicited may be used to classify or otherwise denote the data/matter as being of a specific type. The data add form is preferably generated as a html or XML format document that can be accessed and viewed by a user of a DLMSU 90 by way of a generally available web browser such as, for example, Microsoft Internet Explorer, Mozilla Foxfire or Opera.

Once the data form is submitted to the DLMSU 100, a record is created that is preferably associated with, at a minimum, a classification/type and a LSO product ID before the transaction record/data is added to the Central Data Repository 125. If a new document is being added to the central data repository, a link (example: html or Xml compliant link) to the document is placed in the transaction record so that the document can be easily accessed from the transaction record.

This process is preferably carried out for all transaction records including documents added to the central data repository, as well as all records created to reflect, for example, events, occurrences, scheduled deadlines, reminders, reported events and/or responsive actions, and/or investigations, corrective actions, carried out in connection with a particular drug product.

As data is added to the Central Data Repository 125 a record (transaction record) reflecting the event/data being added to the transactional database 126 (FIG. 4-) of the central data repository 125 is created that includes, but is not limited to, the classification of the data and an associated LSO product is created and added to a the data warehouse database 128 (FIG. 4-).

The DLMS 100 is preferably configured to transform the contents of the transactional database 126 into data warehouse 128 (FIG. 4-). This data warehouse 128 may be used as the source for data reflected/reported by the Summary Dashboard 460 (FIG. 4G). Data warehouse 128 (FIG. 4-) is preferably configured as a data warehouse database and it is used to update the various monitors displayed by the summary dashboard 460.

With reference to FIG. 7B, one embodiment of the DLMS 100 is configured to monitor the transactional database 126 in accordance with predetermined rules in order to identify any recorded events that would trigger action (720). If relevant data is called for by the rules, data relevant to the event will be identified (722). If the rules call for the issuance of an alert or other notification, an alert or notification will be generated and distributed according to applicable rules (724). If a particular response is called for by the rules, a response action plan may be generated and distributed to relevant parties (726). Data contained in the Central Data Repository 125 that may be relevant to the alert may also be provided/identified (728). Times/dates of deadlines, reminders and/or follow-up actions will be calendared in accordance with relevant rules (730). Follow-up reminder or status inquires may be generated and distributed in accordance with calendared times/dates and other applicable rules (732).

The advantages of the present invention can be seen when we consider a scenario where a deviation, in particular an adverse medical event (AE) has been reported to the LSO. More particularly, consider the case where a fatality has been reported in connection with the use of a particular LSO product (Product A) that is manufactured and/or distributed by the LSO.

In the event of a fatality reported in connection with a drug product, certain regulatory authorities such as, for example, the FDA in the United States, require certain prompt and complete actions to be undertaken and completed within a very specific timeframe. If these actions are not undertaken and completed within the specified time frame, the LSO may be subjected to staggering fines or penalties. In the extreme case, the LSO may also be enjoined from further operations until such time as being required action is completed or other measures are taken.

Monitoring Spontaneous Post Marketing Related Records—Once an a deviation, such as an adverse is reported spontaneous post marketing event, such as an adverse event, is reported to the LSO, it is submitted to the DLMS 100 wherein a record reflecting the adverse event is then added to the collective data repository 125. The record may be classified or otherwise typed as a FATAL EVENT. The DLMS 100 is configured to monitor reported adverse events that are added to the collective data repository 125 in accordance with predetermined rules.

Alerts—Where the DLMS 100 determines that a report of a fatal event has been made in relation to, for example, Product A, the DLMS 100 is configured to generate and issue an alert notification to one or more predetermined parties. Typically this alert notification will be directed to, for example, quality-control and/or safety Department personnel, to advise them that a fatality has been reported in connection with Product A. Preferably, the primary alert notification will be issued via e-mail while other supplementary alert notifications may also be provided via, for example, voice message directed to one or more telephone numbers or the automatic generation and issuance of a fax to one or more predetermined fax numbers.

Response Action Plans-Based upon predefined rules stored in the central data repository 125, the DLMS 100 is able to quickly determine that the occurrence of a fatal event report requires certain prompt and specific actions to be taken by the LSO within a predetermined time frame.

In order to aid personnel of the LSO in responding to the report of a fatal event, the DLMS 100 is also configured to generate and include as a part of, or in connection with, the alert notification, a response action plan that sets out the steps that should be followed in order to properly respond to occurrence of a fatal event. The response action plan may be directly set out in the body of the e-mail alert notification or, a link (hyperlink) may be provided in the body of the e-mail alert notification that will allow the recipient of the e-mail alert notification to access and view a recommended response action plan published by the DLMS 100. The published response action plan will preferably be generated by the DLMS 100 in an electronic format suitable for accessing and viewing via a Web browser such as, for example, Microsoft Internet Explorer, Mozilla Foxfire or Opera.

In the case of a fatal event, the response action plan may indicate that a particular regulatory agency must be notified within 48 hours of the report of the fatal event. It may further provide a specific telephone number that should be called within 48 hours in order to report the fatal event to the regulatory agency. Further, it may provide instructions to personnel to obtain the name of the party they speak to what may contact the regulatory agency via telephone and to also note the time of the call is made. The response action plan may also set out that a formal report must be submitted to regulatory authorities in writing and within seven days.

Providing Access to Relevant Data-After determining that Product A is at issue and/or that a particular batch of Product A is specifically at issue, the DLMS 100 may be configured to provide a link to all relevant data contained in the central data repository 125 that concerns Product A and/or a specific batch of Product A (batch number) number may be generated and included in the response action plan.

Calendaring Due Dates, Reminders & Follow-up Inquiries—After an alert notification or response action plane has been issued, the DLMS 100 is configured to calendar specific times and/or dates for reminders, and/or status inquires to be issued and forwarded to responsible personnel and/or their supervisors or management. Upon the calendared time or date, the DLSM 100 is configured to generate and issue a reminder or status inquiry.

Issuance of alerts, response action plan, and/or calendaring of due dates and follow-up is not limited to reports of deviations/adverse events/fatal events. The DMLS 100 may be configured to provide for, as desired or required, the issuance of alerts, response action plan, and/or calendaring of due dates and follow-up for any type of reported event/occurrence (event record) by defining an appropriate business rule, or set of rules, to be applied, or triggered, upon the detection of a predetermined event/occurrence (as indicated by one or more event records stored into the central data repository 125).

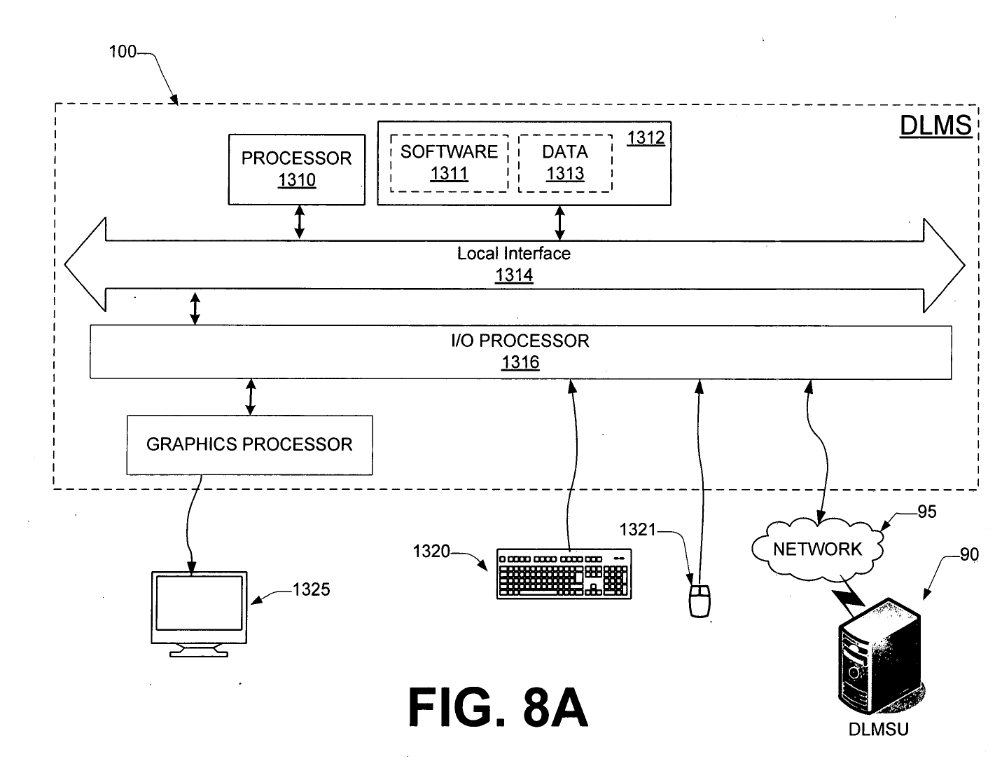

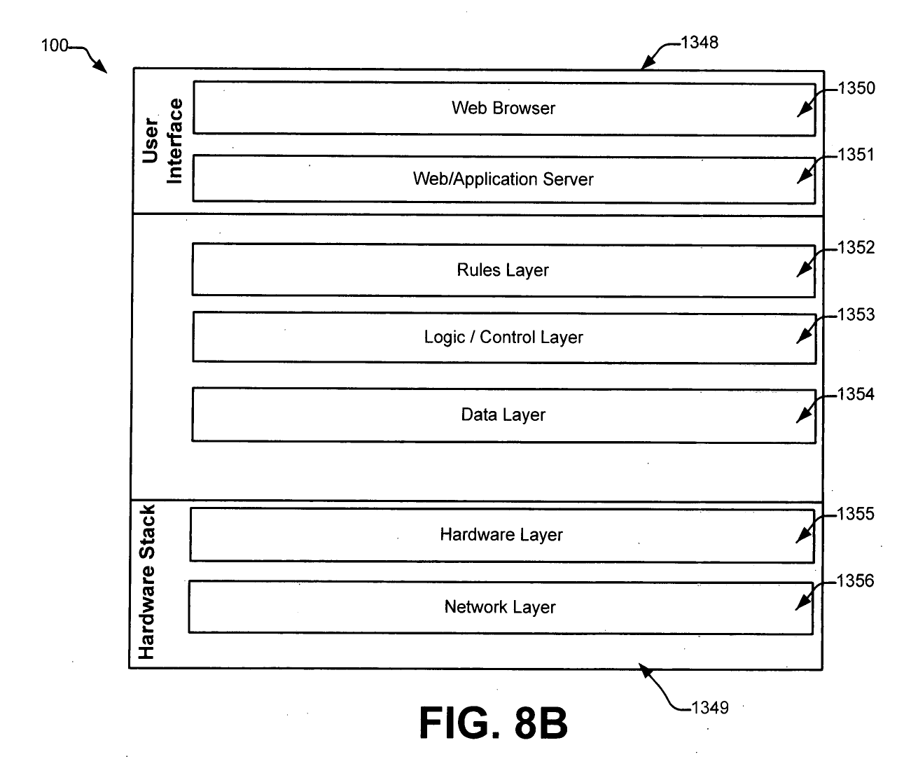

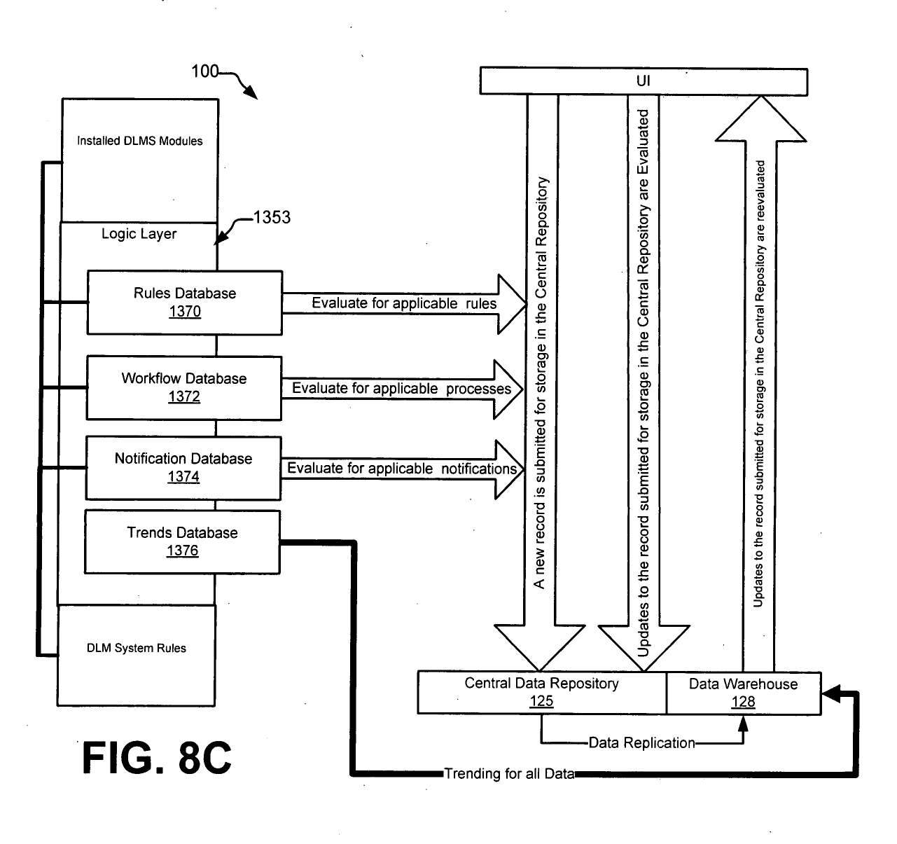

FIG. 8A-FIG. 8C are diagrams depicting possible implementations of a DLMS 100. With reference to the example of FIG. 8A, the DLMS 100 is configured to include a processor 1310 and memory 1312, for storing software 1311 and data 1313. A local interface 1314 is provided to allow the processor 1310 and other components of the DLMS 100 to exchange instructions and/or data. An I/O processor 1316 is provided to receive input from input devices such as keyboard 1320 and pointing device 1321. It also provides for output of data to graphics processor 1318 for generation of an output video or graphics signal to display device 1325. Software 1311 may be configured to carry out any one or more of the functions and processes described herein and/or shown in the flowcharts herein.

The DLMS 100 may be configured to request data from, for example, a DMLS unit. Similarly, a DMLSU 90 may be configured to receive data and/or queries from, for example, the DMLS 100. The DLMS 100 can be implemented in hardware, software, firmware, or a combination thereof. In a preferred embodiment(s), the DMLS 100 is implemented in software or firmware that is stored in a memory and that is executed by a suitable instruction execution system. If implemented in hardware, as in an alternative embodiment, the DLMS 100 can be implemented with any one or a combination of the following technologies, which are all well known in the art: a discrete logic circuit(s) having logic gates for implementing logic functions upon data signals, an application specific integrated circuit having appropriate logic gates, a programmable gate array(s) (PGA), a fully programmable gate array (FPGA), etc. Software

A DLMSU 90 can be implemented in hardware, software, firmware, or a combination thereof. In a preferred embodiment(s), the DLMSU 90 is implemented in software or firmware that is stored in a memory and that is executed by a suitable instruction execution system. If implemented in hardware, as in an alternative embodiment, the DLMSU 90 can be implemented with any one or a combination of the following technologies, which are all well known in the art: a discrete logic circuit(s) having logic gates for implementing logic functions upon data signals, an application specific integrated circuit having appropriate logic gates, a programmable gate array(s) (PGA), a fully programmable gate array (FPGA), etc.

With reference to FIG. 8B the DMSL 100 may be implemented to include a user interface 1348, that incorporates, for example, a web browser 1350 and a web/application server 1351 for publishing document, forms reports and other for user access. A rules layer 1352 is provided that defines a common set of business rules to be applied to all data. A logic/control layer 1353 is provided that defines various processes/operations that should be carried out in connection with or in response to the data, based upon the predefined business rules. A data layer 1354 is provided for accommodating data submitted for incorporation into the central data repository 125. A hardware stack 1349 is provided that includes a hardware layer 1355 and a network layer 1356. The hardware stack is configured to allow the DLMS to interface with hardware and network components.

FIG. 8C is a diagram depicting a further implementation of the DLMS 100. A logic later 1352 is provided. The logic layer is configured to carry out certain processes in relation to data to be added to the central data repository 125. These processes include, evaluating the data to determine if certain rules apply to the data; evaluating the data to determine if there are responsive processes that are required in connection with the data; and evaluating the data to determine if there are notifications that should be issued in connection with certain data. Trending functionality is provided. The data warehouse 128 is monitored to determine a current status of various predefined business/product related aspects/variables.

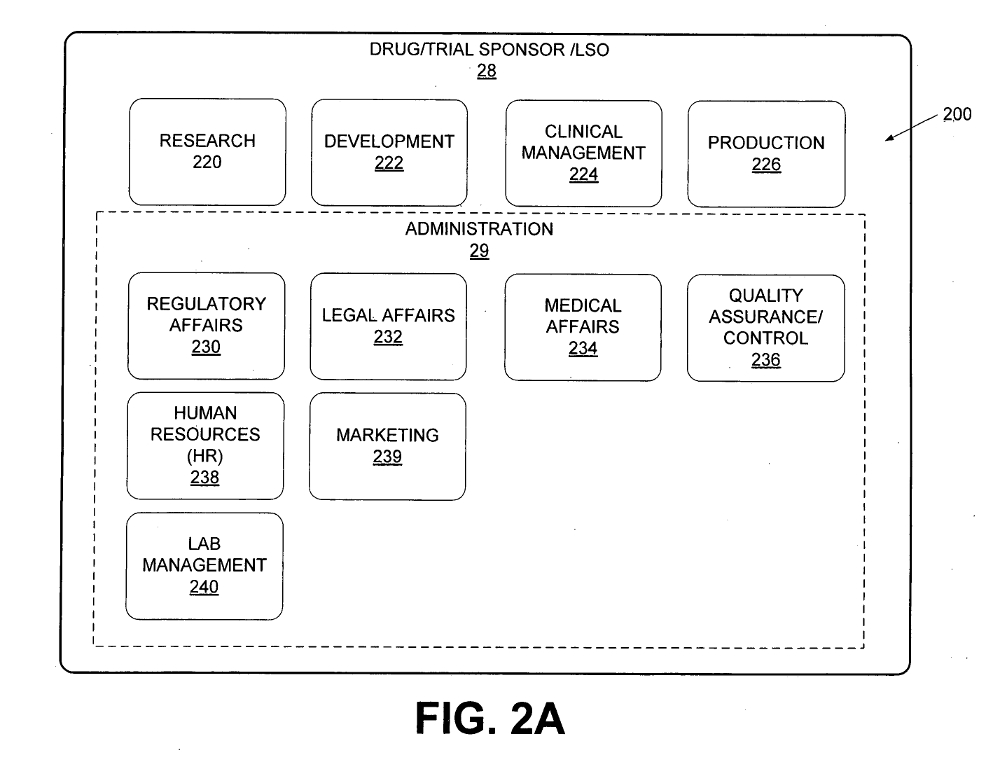

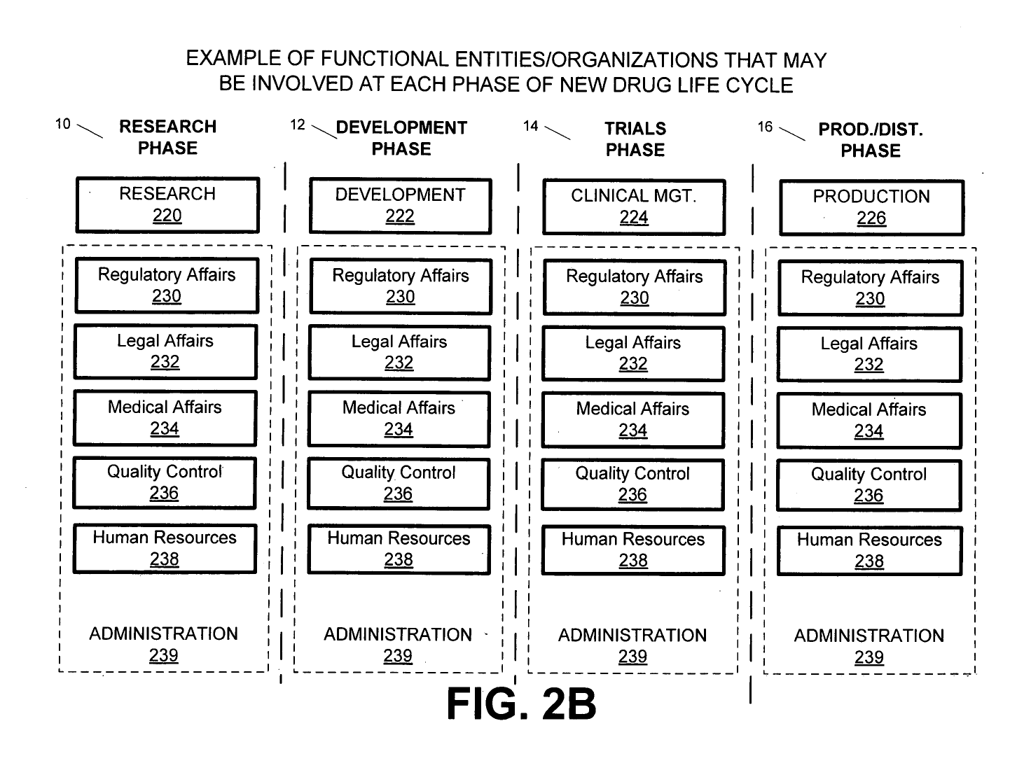

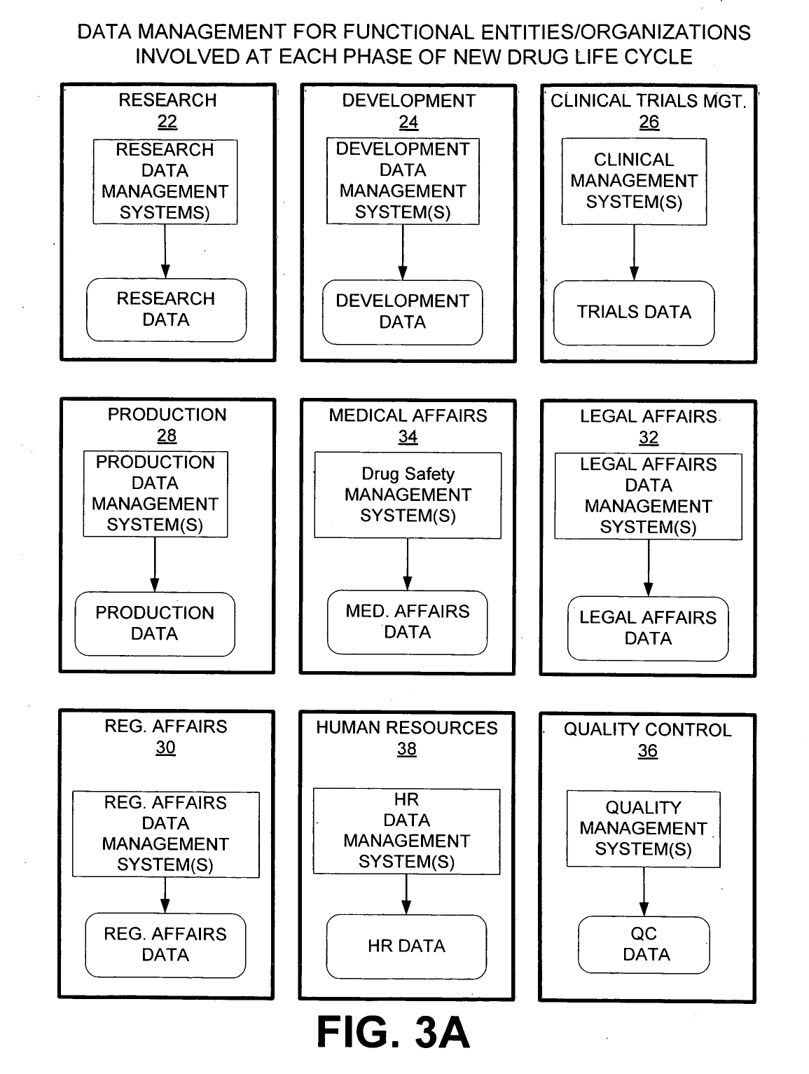

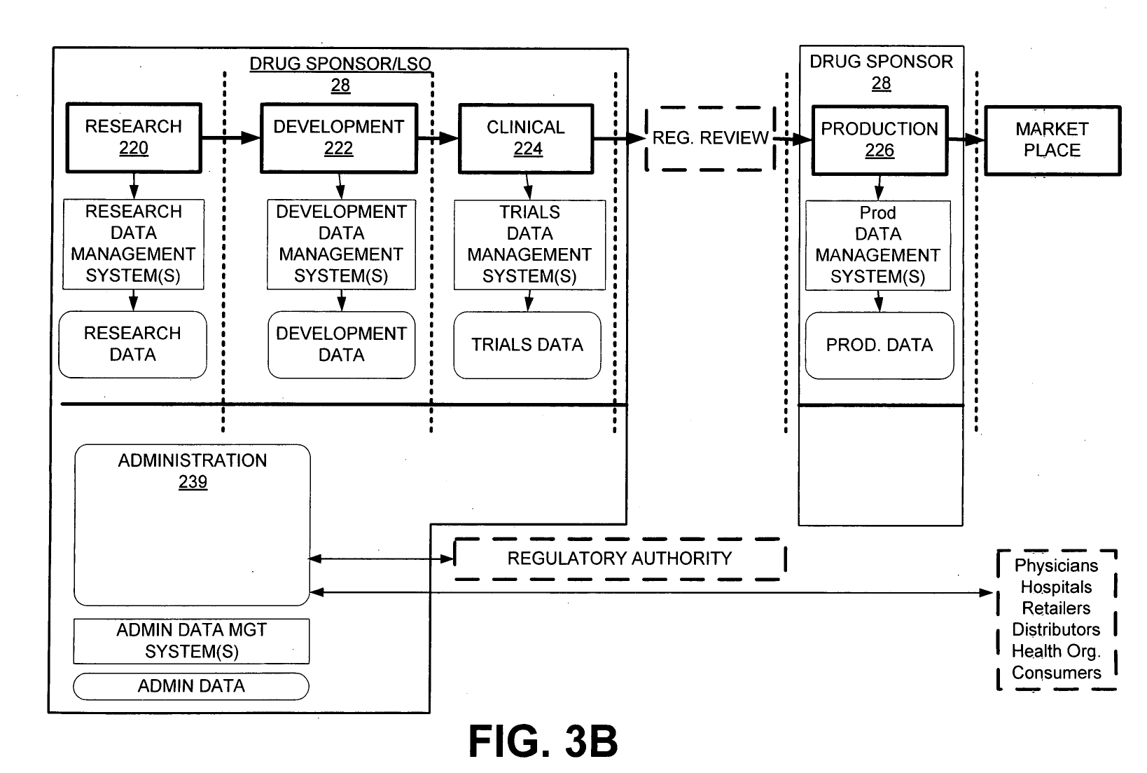

The flow charts of FIG. 4B, 4H, 4J, 5, 6A, 6B, 6C, 7A and/or 7C show the architecture, functionality, and operation of possible implementations of the software 258 (FIG. 2C). In this regard, each block represents a module, segment, or portion of code, which comprises one or more executable instructions for implementing the specified logical function(s). It should also be noted that in some alternative implementations, the functions noted in the blocks may occur out of the order noted in the flowcharts. For example, two blocks shown in succession in the flowcharts may in fact be executed substantially concurrently or the blocks may sometimes be executed in the reverse order, depending upon the functionality involved. The software program stored as software 305, which comprises a listing of executable instructions (either ordered or non-ordered) for implementing logical functions, can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can contain, store, communicate, propagate, or transport the program for use by or in connection with the instruction execution system, apparatus, or device. The computer-readable medium can be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection (electronic) having one or more wires, a portable computer diskette (magnetic), a random access memory (RAM) (magnetic or non-magnetic), a read-only memory (ROM) (magnetic or non-magnetic), an erasable programmable read-only memory (EPROM or Flash memory), an optical fiber (optical), and a portable compact disc read-only memory (CDROM) (optical or magneto-optical). Note that the computer-readable medium could even be paper or another suitable medium upon which the program is printed, as the program can be electronically captured, via for instance, optical scanning of the paper or other medium, then compiled, interpreted or otherwise processed in a suitable manner, if necessary, and then stored in a computer memory.

It will be recognized by those skilled in the art, that while certain aspects of the invention have been described in terms of hardware, it is possible and fully anticipated that such aspects can be implemented in software, and vice-a-versa. All such variations or implementations are fully contemplated by the present invention and are intended to full within the scope of the invention.

It should be emphasized that the above-described embodiments of the present invention, particularly, any “preferred” embodiments, are merely possible examples of implementations, merely set forth for a clear understanding of the principles of the invention. Many variations and modifications may be made to the above-described embodiment(s) of the invention without departing substantially from the spirit, principles and scope of the invention. All such modifications and variations are fully intended to be included herein within the scope of the present invention and protected by the following claims.

What is claimed is:

1. A Drug life cycle management system comprising a central data repository for storing data related to a drug product.

2. The Drug life cycle management system of claim 1, further comprising control module configured to analyze the data in accordance with a common set of predetermined rules.

3. The Drug life cycle management system of claim 1 wherein the data comprises all data generated by business entities involved in the life cycle of the drug product.

4. The Drug life cycle management system of claim 2 wherein the control module is further configured to invoke the issuance of a notification in accordance with the common set of predetermined rules.

5. The Drug life cycle management system of claim 4 wherein the control module is further configured to issue a response action plan in accordance with the common set of predetermined rules.

6. The Drug life cycle management system of claim 1 further comprising a summary dashboard configured to provide a monitor indicative of a current status of a predetermined factor related to the drug product.

7. A drug life cycle management system configured to carry out the steps of:
Receiving data related to a drug product from a business entity involved in the life cycle of a drug product; and
Storing the data into a central data repository.

8. The drug life cycle management system of claim 7 further configured to apply a common set of business rules to the data.

9. The drug life cycle management system of claim 8 further configured to carry out the step of invoking issuance of a notification based upon the predetermined set of common business rules.

10. The drug life cycle management system of claim 7 further configured to carry out the step of issuing a response action plan based upon the predetermined set of common business rules.

11. The drug life cycle management system of claim 7 further configured to carry out the step of publishing a summary dash board indicative of a current status of a predetermined factor related to the drug product.
